(19) United States
(12) Patent Application Publication
(10) Pub. No.: US 2024/0125765 A1
(43) Pub. Date: Apr. 18, 2024

(54) A METHOD FOR SELECTION OF CRYOPRESERVED CORD BLOOD UNITS FOR THE MANUFACTURE OF ENGINEERED NATURAL KILLER CELLS WITH ENHANCED POTENCY AGAINST CANCER

(71) Applicant: BOARD OF REGENTS, THE UNIVERSITY OF TEXAS SYSTEM, Austin, TX (US)

(72) Inventors: Katy REZVANI, Houston, TX (US); David MARIN COSTA, Houston, TX (US); Elizabeth SHPALL, Houston, TX (US)

(21) Appl. No.: 18/547,401

(22) PCT Filed: Mar. 16, 2022
(86) PCT No.: PCT/US2022/020572

§ 371 Date: Aug. 22, 2023

Related U.S. Application Data
(60) Provisional application No. 63/243,669, filed on Sep. 13, 2021.
(60) Provisional application No. 63/164,379, filed on Mar. 22, 2021.

(51) Int. Cl. G01N 33/50; A61K 39/00; C12N 5/0783

(52) U.S. Cl. CPC G01N 33/5005; A61K 39/4613; A61K 39/4631; C12N 5/0646; C12N 2510/00

(57) ABSTRACT

Embodiments of the disclosure concern methods and compositions related to optimization of selection of cord blood units to produce immune cells, such as NK cells, for adoptive cell therapy use. In specific embodiments, particular characteristics of the cord blood units and/or characteristics of cells derived therefrom are analyzed. When a threshold measurement for one or more characteristics is met for the cord blood unit(s) and/or characteristics of cells derived therefrom, the cord blood unit(s) are utilized as a source for production of immune cells. Specific characteristics for measurement include cord blood cell viability, total nuclear cell recovery, and nucleated red blood cell content, each prior to cryopreservation, and optionally cytotoxicity and/or expansion of immune cells subsequent to cryopreservation.

This application claims priority to U.S. Provisional Patent Application Ser. No. 63/164,379, filed Mar. 22, 2021 and to U.S. Provisional Patent Application Ser. No. 63/243,669, filed Sep. 13, 2021, both of which applications are incorporated by reference herein in their entirety.

### TECHNICAL FIELD

Embodiments of the disclosure concern at least the technical fields of cell biology, molecular biology, immunology, and medicine.

### BACKGROUND

Umbilical cord blood derived natural killer (NK) cells modified to express a CAR are an effective therapy against cancer. Indeed, umbilical cord derived NK cells can be modified (either through genetic or non-genetic methods) to treat multiple malignancies and infections. Cryopreserved cord blood units are readily available in biobanks (as they are used as a source of cells for stem cell transplantation) and can provide sufficient numbers of NK cells to manufacture multiple cell therapy products for clinical use. The alternative to the use of cord blood units as a source of NK cells is to obtain cells from healthy donors by the means of leukoapheresis. This procedure is complex and it is not exempt of risk to the donor. The clinical efficacy of an NK cell product is heavily influenced by the characteristics of the cryopreserved cord units. The present disclosure satisfies a long-felt need in the art of procuring suitable cells for cell therapy.

### BRIEF SUMMARY

The present disclosure is directed to methods and compositions related to cell therapy for an individual. The cell therapy may be of any kind, but in specific embodiments the cell therapy comprises adoptive cell therapy with immune cells, including at least immune cells that eventually may be modified prior to administration to an individual in need of the cells. In particular embodiments, the disclosure concerns identification of cord blood units particularly suited to produce effective immune cells for adoptive cell therapy for an individual, including that is more effective than selection of cord blood in the absence of the identification.

The present disclosure concerns a multi-part strategy to identify cord blood units that are most likely to produce highly efficacious immune cell therapy products for the treatment of patients, including treatment for any kind of medical condition, at least such as cancer or infection of any kind. The disclosure provides a set of selection criteria including criteria that is: (i) prior to the cryopreservation of the cord blood unit, (ii) post thaw and at the start of immune cell manufacture, such as in a GMP facility, and (iii) immune cell characteristics during and at the end of manufacture.

Particular embodiments include methods of selecting a cord blood composition, comprising the steps of measuring prior to cryopreservation or use of the cord blood composition: (a) cord blood cell viability; (b) optionally total mononuclear cell (TNC) recovery; (c) nucleated red blood cell (NRBC) content; (d) weight of the baby from which the cord blood is derived; (e) race of the biological mother and/or biological father of the baby from which the cord blood is derived; (f) optionally gestational age of the baby from which the cord blood is derived; (g) optionally intra utero collection of the cord blood (although extra utero or a combination of intra utero and extra utero may be used in any method of the disclosure); (h) optionally a biologically male baby from which the cord blood is derived; (i) optionally a pre-process volume (volume of the cord blood collected plus anticoagulant (one example is 35 ml citrate phosphate dextrose (CPD)) ≤120 mL; (j) optionally cells of the extracted cord blood are >0.4% CD34+; and optionally measuring subsequent to cryopreservation (k) cytotoxicity of immune cells derived from the cord blood composition following thawing; and (1) fold expansion of the immune cells derived from the cord blood (including NK cells) during culture after thawing. In specific embodiments, the criteria meet a quantitate threshold for at least one of the characteristics.

In specific cases, the following one or more criteria are met: (a) cord blood cell viability greater than or equal to 98% or 99%; (b) optional total mononuclear cell (TNC) recovery is greater than or equal to 76.3%; and (c) nucleated red blood cell (NRBC) content less than or equal to 7.5×10⁷ or 8.0×10⁷ or any amount therebetween.

Embodiments of the disclosure encompass methods of selecting a cord blood composition, comprising the steps of: measuring prior to cryopreservation of the cord blood composition: (a) cord blood cell viability; (b) optionally total mononuclear cell (TNC) recovery; (c) nucleated red blood cell (NRBC) content; (d) weight of the baby from which the cord blood is derived; (e) race of the biological mother and/or biological father of the baby from which the cord blood is derived; (f) optionally gestational age of the baby from which the cord blood is derived; (g) optionally intra utero collection of the cord blood (although extra utero or a combination of intra utero and extra utero may be used in any method of the disclosure); (h) optionally a biologically male baby from which the cord blood is derived; (i) optionally a pre-process volume (volume of the cord blood collected plus anticoagulant (35 ml CPD)) ≤120 mL; (j) optionally, cells of the extracted cord blood are >0.4% CD34+; and measuring subsequent to cryopreservation (d) optionally cytotoxicity of immune cells derived from the cord blood composition following thawing. In specific embodiments, the immune cells are natural killer (NK) cells. Methods may further comprise the step of expanding the NK cells and/or modifying the NK cells. In some cases, the NK cells are modified to express one or more non-endogenous gene products, such as one or more non-endogenous receptors (such as one or more chimeric receptors, including one or more chimeric antigen receptors and/or one or more non-natural T-cell receptors). In some cases, the non-endogenous gene product comprises one or more non-endogenous receptors, one or more cytokines, one or more chemokines, one or more enzymes, or a combination thereof. The NK cells may be modified to have disruption of expression of one or more endogenous genes in the NK cells.

In one embodiment, there is a method of selecting a cord blood composition, comprising the steps of: identifying a cord blood composition that, prior to cryopreservation, is determined to have one or more of the following: (a) cord blood cell viability greater than or equal to 98% or 99%; (b) optionally total mononuclear cell (TNC) recovery is greater than or equal to 76.3%; (c) nucleated red blood cell (NRBC) content less than or equal to 7.5×10⁷ or 8.0×10⁷ or any amount therebetween; (d) weight of the baby from which the cord blood is derived is greater than 3650 grams; (e) race of the biological mother and/or biological father of the baby from which the cord blood is derived is Caucasian; (f) optionally gestational age of the baby from which the cord blood is derived is less than or equal to about 38 weeks; (g) optionally intra utero collection of the cord blood (although extra utero or a combination of intra utero and extra utero may be used in any method of the disclosure); (h) optionally a biologically male baby from which the cord blood is derived; (i) optionally a pre-process volume (volume of the cord blood collected plus anticoagulant (35 ml CPD)) ≤120 mL; (j) optionally, cells of the extracted cord blood are >0.4% CD34+; and optionally (k) measuring cytotoxicity of immune cells derived from the cord blood composition following thawing. In some cases, the cord blood composition prior to cryopreservation is determined to have at least (a) and (b); determined to have (b) and (c); determined to have (a) and (c); or determined to have (a), (b), and (c).

In specific cases, the cord blood cell viability in (a) is greater than or equal to 98.1, 98.2, 98.3, 98.4, 98.5, 98.6, 98.7, 98.8, 98.9, 99.0, 99.1, 99.2, 99.3, 99.4, 99.5, 99.6, 99.7, 99.8, or 99.9%. In specific cases, the TNC recovery in (b) is greater than or equal to 77, 78, 79, 80, 81, 82, 83, 84, 85, 86, 87, 88, 89, 90, 91, 92, 93, 94, 95, 96, 97, 98, or 99%. In specific cases, the NRBC content is less than or equal to 8.0×10⁷, 7.9×10⁷, 7.8×10⁷, 7.7×10⁷, 7.6×10⁷, 7.5×10⁷, 7.0×10⁷, 6.0×10⁷, 5.0×10⁷, 4.0×10⁷, 3.0×10⁷, 2.0×10⁷, 1.0×10⁷, 9.0×10⁶, 8.0×10⁶, 7.0×10⁶, 6.0×10⁶, 5.0×10⁶, 4.0×10⁶, 3.0×10⁶, 2.0×10⁶, 1.0×10⁶, 9.0×10⁵, 8.0×10⁵, 7.0×10⁵, 6.0×10⁵, 5.0×10⁵, 4.0×10⁵, 3.0×10⁵, 2.0×10⁵, 1.0×10⁵, 9.0×10⁴, 8.0×10⁴, 7.0×10⁴, 6.0×10⁴, 5.0×10⁴, 4.0×10⁴, 3.0×10⁴, 2.0×10⁴, 1.0×10⁴, 9.0×10³, 8.0×10³, 7.0×10³, 6.0×10³, 5.0×10³, 4.0×10³, 3.0×10³, 2.0×10³, 1.0×10³, 9.0×10², 8.0×10², 7.0×10², 6.0×10², 5.0×10², 4.0×10², 3.0×10², 2.0×10², 1.0×10², and so forth. In specific cases, the weight of the baby from which the cord blood is derived is greater than 3650 grams. In specific cases, the race of the biological mother from which the cord blood is derived is Caucasian and/or biological father of the baby from which the cord blood is derived is Caucasian. In specific embodiments, the gestational age of the baby from which the cord blood is derived is less than or equal to about 38 weeks. In certain embodiments, the cord blood may be obtained by any suitable method, but in specific embodiments it is obtained in utero, extra utero, or both, although in particular cases it is obtained in utero only. In certain embodiments, the volume of the extracted cord blood in addition to a volume of about 35 mL of anticoagulant is ≤120 mL, such that the volume of the extracted cord blood is no greater than about 85, 84, 83, 82, 81, 80, 79, 78, 77, 76, 75, 74, 73, 72, 71, 70, 69, 68, 67, 66, 65, 64, 63, 62, 61, 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, or 30 mL or less in volume.

Any method encompassed herein may further comprise the step of deriving immune cells from the thawed cord blood composition. The immune cells may be NK cells, invariant NK cells, NK T cells, T cells B cells, monocytes, granulocytes, myeloid cells neutrophils, eosinophils, basophils, mast cells, monocytes, macrophages, dendritic cells, stem cells, or a mixture thereof. In specific cases, the immune cells derived from the cord blood composition following thawing are NK cells and the cytotoxicity is greater than or equal to 66.7%. The cytotoxicity may be greater than or equal to 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99%.

In some embodiments, the cord blood is derived from a fetus or infant at less than or equal to 39 or 38 weeks of gestational age. The cord blood may be derived from a fetus or infant at less than or equal to 39, 38, 37, 36, 35, 34, 33, 32, 31, 30, 29, 28, 27, 26, 25, or 24 weeks or less of gestational age. In some cases, the method further comprises determining viability of cord blood cells following thawing. In specific aspects, the viability of cord blood cells following thawing is greater than or equal to 86.5%, such as greater than or equal to 87, 88, 89, 90, 91, 92, 93, 94, 95, 96, 97, 98, or 99%.

When the immune cells derived from the thawed cord blood composition are NK cells, they may be expanded. The expansion parameters may or may not be determined on a case-by-case basis. The expansion may be quantified after a particular number of days in culture, such as between day 0 and day 15 and any range therebetween. The fold of expansion by the cells may be of any suitable quantity, such as at least, or greater than about, 3-fold, 5-fold, 7-fold, 10-fold, 20-fold, 25-fold, 50-fold, 75-fold, 100-fold, 125-fold, 150-fold, 175-fold, 200-fold, 225-fold, 250-fold, 275-fold, 300-fold, 325-fold, 350-fold, 375-fold, 400-fold, 425-fold, 450-fold, 475-fold, 500-fold, and so forth. In some cases, the expansion of the NK cells between days 0 and 6 in culture is greater than or equal to 7-fold. In some cases, the expansion of the NK cells between days 6 and 15 in culture is greater than or equal to 10-fold. In specific cases, the expansion is between 0 and 15 days or 6 and 15 days or 0 and 6 days (and any range therebetween) and has a greater than 70-fold expansion. In specific cases, the expansion is between 0 and 15 days (and any range therebetween) and has a greater than 450-fold expansion. Ranges of days of expansion with any fold level may include 0-15, 0-14, 0-13, 0-12, 0-11, 0-10, 0-9, 0-8, 0-7, 0-6, 0-5, 0-4, 0-3, 0-2, 0-1, 1-15, 1-14, 1-13, 1-12, 1-11, 1-10, 1-9, 1-8, 1-7, 1-6, 1-5, 1-4, 1-3, 1-2, 2-15, 2-14, 2-13, 2-12, 2-11, 2-10, 2-9, 2-8, 2-7, 2-6, 2-5, 2-4, 2-3, 3-15, 3-14, 3-13, 3-12, 3-11, 3-10, 3-9, 3-8, 3-7, 3-6, 3-5, 3-4, 4-15, 4-14, 4-13, 4-12, 4-11, 4-10, 4-9, 4-8, 4-7, 4-6, 4-5, 5-15, 5-14, 5-13, 5-12, 5-11, 5-10, 5-9, 5-8, 5-7, 5-6, 6-15, 6-14, 6-13, 6-12, 6-11, 6-10, 6-9, 6-8, 6-7, 7-15, 7-14, 7-13, 7-12, 7-11, 7-10, 7-9, 7-8, 8-15, 8-14, 8-13, 8-12, 8-11, 8-10, 8-9, 9-15, 9-14, 9-13, 9-12, 9-11, 9-10, 10-15, 10-14, 10-13, 10-12, 10-11, 11-15, 11-14, 11-13, 11-12, 12-15, 12-14, 12-13, 13-15, 13-14, 14-15, and so forth.

The NK cells may be modified, such as modified to express one or more non-endogenous gene products, such as a non-endogenous receptor, including a chimeric receptor, such as a chimeric antigen receptor or non-endogenous receptor is a non-natural T-cell receptor. In some cases, the non-endogenous gene product comprises one or more non-endogenous receptors, one or more cytokines, one or more chemokines, one or more enzymes, or a combination thereof. In specific cases, immune cells derived from the thawed cord blood composition are modified to have disruption of expression of one or more endogenous genes in the cells.

In a specific case, the cord blood cell viability is greater than 98% or 99%, the TNC recovery is greater than 76.3%, and the NRBC content is lower than 7.5×10⁷ or 8.0×10⁷ or any range therebetween, including 7.5×10⁷-8.0×10⁷, 7.5×10⁷-7.9; 7.5×10⁷-7.8×10⁷; 7.5×10⁷-7.7×10⁷; 7.5×10⁷-7.6×10⁷; 7.6×10⁷-8.0×10⁷; 7.6×10⁷-7.9×10⁷; 7.6×10⁷-7.8×10⁷; 7.6×10⁷-7.7×10⁷; 7.7×10⁷-8.0×10⁷; 7.7×10⁷-7.9×10⁷; 7.7×10⁷-7.8×10⁷; 7.8×10⁷-8.0×10⁷; 7.8×10⁷-7.9×10⁷; 7.9×10⁷-8.0×10⁷ In specific embodiments, the cord blood is derived from a fetus or infant at less than or equal to 39 or 38 weeks of gestational age, the viability of cord blood cells following thawing is greater than or equal to 86.5% (and this is optional), the expansion of the NK cells between days 0 and 6 in culture is greater than or equal to 3-fold, and the expansion of the NK cells between days 6 and 15 in culture is greater than or equal to 100-fold, and the expansion of the NK cells between days 0 and 15 is greater than or equal to 900-fold. In specific cases, the expansion is between 6 and 15 days and has a greater than 70-fold expansion. In specific cases, the expansion is between 0 and 15 days and has a greater than 450-fold expansion.

Embodiments of the disclosure comprise cord blood compositions identified by any one of the methods encompassed herein. The composition may be comprised in a pharmaceutically acceptable carrier. The composition may be formulated with one or more cryoprotectants.

Embodiments of the disclosure comprise compositions comprising a population of immune cells derived from any method encompassed herein.

In some embodiments there is a method of predicting efficacy of immune cells for therapy, comprising measuring one or more cord blood compositions having not been frozen for the following: (a) cord blood cell viability; (b) optionally total mononuclear cell (TNC) recovery; (c) nucleated red blood cell (NRBC) content; (d) weight of the baby from which the cord blood is derived; (e) race of the biological mother and/or biological father of the baby from which the cord blood is derived is Caucasian; (f) optionally gestational age of the baby from which the cord blood is derived; wherein the immune cells are efficacious for therapy when the cord blood composition comprises one or more of the following characteristics: (a) cord blood cell viability greater than or equal to 98% or 99%; (b) the optional total mononuclear cell (TNC) recovery is greater than or equal to 76.3%; (c) nucleated red blood cell (NRBC) content less than or equal to 8.0×10⁷; (d) weight of the baby from which the cord blood is derived is greater than 3650 grams; (e) race of the biological mother and/or biological father of the baby from which the cord blood is derived is Caucasian; (f) optionally gestational age of the baby from which the cord blood is derived is less than or equal to about 38 weeks. The method may further comprise the step of freezing the one or more blood compositions. The method may further comprise measuring upon thawing (d) cytotoxicity of immune cells derived from the cord blood composition. In some cases, the cytotoxicity is greater than or equal to 66.7%.

The foregoing has outlined rather broadly the features and technical advantages of the present disclosure in order that the detailed description that follows may be better understood. Additional features and advantages will be described hereinafter which form the subject of the claims herein. It should be appreciated by those skilled in the art that the conception and specific embodiments disclosed may be readily utilized as a basis for modifying or designing other structures for carrying out the same purposes of the present designs. It should also be realized by those skilled in the art that such equivalent constructions do not depart from the spirit and scope as set forth in the appended claims. The novel features which are believed to be characteristic of the designs disclosed herein, both as to the organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present disclosure.

### BRIEF DESCRIPTION OF THE DRAWINGS

For a more complete understanding of the present disclosure, reference is now made to the following descriptions taken in conjunction with the accompanying drawings.

FIG. 1. Pre-freezing CBU characteristics predict for clinical response directed to cell viability.

FIG. 2. Pre-freezing CBU characteristics predict for clinical response directed to total mononuclear cell (TNC) recovery.

FIG. 3. Pre-freezing CBU characteristics predict for clinical response directed to reduction of nucleated red blood cell (NRBC) content.

FIG. 4. Three CBU characteristics are independent predictors of response in a multivariate model when adjusted by the patient clinical characteristics.

FIG. 5. Number of CBU favorable characteristics at 30 days response (crosstabulation).

FIG. 6. Killing of Raji tumor cells by non-transduced NK cells (from frozen CB) is an independent predictor for clinical response (measured by ⁵¹Cr release assay).

FIGS. 7A-7B. The use of additional parameters to improve the prediction for clinical response, directed to using cell viability >98%; TNC recovery >76.3%; and NRBC content (7A) vs. using those three in addition to gestational age <39 weeks; cord blood post thaw viability >86.5%; NK cell expansion between days 0 and 6 in culture greater than or equal to 3-fold; NK cell expansion between days 6 and 15 in culture greater than or equal to 100-fold; and/or NK cell expansion between days 0 and 15 in culture greater than or equal to 900-fold.

FIG. 8A shows cell viability of cord blood units as measured by flow cytometry can be used to predict for the achievement of complete response and identifies 99% as the optimal cut-off to predict responses. FIG. 8B shows the +30 overall response (PR/CR) and complete responses (CR) according to the cell viability of the cord units. Patients who received cell products derived from CBUs with viability ≥99% have statistically significant better clinical responses than patients who were treated with cell products derived from CBUs with lower viability.

FIG. 9 provides demonstration of nucleated red blood cell count of cord blood units that predicts for the achievement of clinical responses.

FIG. 10A provides race information as it relates to the selected cord blood units and therapy response. FIG. 10B shows the day 30 responses according to the pre-freezing CBU viability and CBU race. Patients who received a cell product derived from CBUs that had a pre-freezing viability ≥99% and were of Caucasian race had an statistically significant CR rate that patients who received cell product derived from CBUs with a pre-freezing viability ≥99% but were not of Caucasian race.

FIG. 11 demonstrates baby weight for the cord blood as it relates to success of therapy.

FIGS. 12A-12C show that selection of CBU based on four particular criteria is the major factor determining patient response.

FIG. 13 provides validation in an independent sample of 19 patients treated with a different NK cell product.

FIG. 14 demonstrates that adding additional characteristics improves the predictive power of the model.

### DETAILED DESCRIPTION

### I. Examples of Definitions

As used herein the specification, “a” or “an” may mean one or more. As used herein in the claim(s), when used in conjunction with the word “comprising,” the words “a” or “an” may mean one or more than one. Some embodiments of the disclosure may consist of or consist essentially of one or more elements, method steps, and/or methods of the disclosure. It is contemplated that any method or composition described herein can be implemented with respect to any other method or composition described herein and that different embodiments may be combined.

The use of the term “or” in the claims is used to mean “and/or” unless explicitly indicated to refer to alternatives only or the alternatives are mutually exclusive, although the disclosure supports a definition that refers to only alternatives and “and/or.” For example, “x, y, and/or z” can refer to “x” alone, “y” alone, “z” alone, “x, y, and z,” “(x and y) or z,” “x or (y and z),” or “x or y or z.” It is specifically contemplated that x, y, or z may be specifically excluded from an embodiment. As used herein “another” may mean at least a second or more. The terms “about”, “substantially” and “approximately” mean, in general, the stated value plus or minus 5%.

Throughout this specification, unless the context requires otherwise, the words “comprise”, “comprises” and “comprising” will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are optional and may or may not be present depending upon whether or not they affect the activity or action of the listed elements.

The term “cord blood composition” or “cord blood unit” as used herein refers to a volume of cord blood originally obtained from a placenta and/or in an attached umbilical cord after childbirth. The cord blood unit or cord blood composition may or may not be stored in a storage facility following its collection. In some cases, the cord blood unit or cord blood composition contains blood that is derived from a single individual, whereas in alternative cases the cord blood unit or cord blood composition is a mixture from multiple individuals.

The term “cryopreservation” as used herein refers to the process of cooling and storing cells at a temperature below the freezing point. In specific examples, the temperature for cryopreservation is at least as low as −80° C. The cryopreservation may or may not include addition of one or more cryoprotectants to the cells prior to freezing. Examples of cryoprotectants include Dimethyl Sulfoxide (DMSO), hetastarch, Dextran 40, or a combination thereof. In one specific example, one may utilize 6% hetastarch in 0.9% sodium chloride in 5 ml of 55% Dimethyl Sulfoxide/5% Dextran 40 in 0.9% sodium chloride.

As used herein, a “disruption” of a gene refers to the elimination or reduction of expression of one or more gene products encoded by the subject gene in a cell, compared to the level of expression of the gene product in the absence of the disruption. Exemplary gene products include mRNA and protein products encoded by the gene. Disruption in some cases is transient or reversible and in other cases is permanent. Disruption in some cases is of a functional or full length protein or mRNA, despite the fact that a truncated or non-functional product may be produced. In some embodiments herein, gene activity or function, as opposed to expression, is disrupted. Gene disruption is generally induced by artificial methods, i.e., by addition or introduction of a compound, molecule, complex, or composition, and/or by disruption of nucleic acid of or associated with the gene, such as at the DNA level. Exemplary methods for gene disruption include gene silencing, knockdown, knockout, and/or gene disruption techniques, such as gene editing. Examples include antisense technology, such as RNAi, siRNA, shRNA, and/or ribozymes, which generally result in transient reduction of expression, as well as gene editing techniques which result in targeted gene inactivation or disruption, e.g., by induction of breaks and/or homologous recombination. Examples include insertions, mutations, and deletions. The disruptions typically result in the repression and/or complete absence of expression of a normal or “wild type” product encoded by the gene. Exemplary of such gene disruptions are insertions, frameshift and missense mutations, deletions, knock-in, and knock-out of the gene or part of the gene, including deletions of the entire gene. Such disruptions can occur in the coding region, e.g., in one or more exons, resulting in the inability to produce a full-length product, functional product, or any product, such as by insertion of a stop codon. Such disruptions may also occur by disruptions in the promoter or enhancer or other region affecting activation of transcription, so as to prevent transcription of the gene. Gene disruptions include gene targeting, including targeted gene inactivation by homologous recombination.

The term “engineered” “or “engineering” as used herein refers to an entity that is generated by the hand of man (or the process of generating same), including a cell, nucleic acid, polypeptide, vector, and so forth. In at least some cases, an engineered entity is synthetic and comprises elements that are not naturally present or configured in the manner in which it is utilized in the disclosure. With respect to cells, the cells may be engineered because they have reduced expression of one or more endogenous genes and/or because they express one or more heterologous genes (such as synthetic antigen receptors and/or cytokines), in which case(s) the engineering is all performed by the hand of man. With respect to an antigen receptor, the antigen receptor may be considered engineered because it comprises multiple components that are genetically recombined to be configured in a manner that is not found in nature, such as in the form of a fusion protein of components not found in nature so configured.

The term “heterologous” as used herein refers to being derived from a different cell type or a different species than the recipient. In specific cases, it refers to a gene or protein that is synthetic and/or not from an NK cell. The term also refers to synthetically derived genes or gene constructs. The term also refers to synthetically derived genes or gene constructs. For example, a cytokine may be considered heterologous with respect to a NK cell even if the cytokine is naturally produced by the NK cell because it was synthetically derived, such as by genetic recombination, including provided to the NK cell in a vector that harbors nucleic acid sequence that encodes the cytokine.

The term “immune cell” as used herein refers to a cell that is part of the immune system and helps the body fight infections and other diseases. Immune cells include natural killer cells, invariant NK cells, NK T cells, T cells of any kind (e.g., regulatory T cells, CD4.sup.+ T cells, CD8.sup.+ T cells, or gamma-delta T cells), B cells, monocytes, granulocytes, myeloid cells neutrophils, eosinophils, basophils, mast cells, monocytes, macrophages, dendritic cells, and/or stem cells (e.g., mesenchymal stem cells (MSCs) or induced pluripotent stem (iPSC) cells). Also provided herein are methods of producing and engineering the immune cells following selection of the appropriate cord blood unit, as well as methods of using and administering the cells for adoptive cell therapy, in which case the cells may be autologous or allogeneic with respect to the source of the cord blood and the recipient of the cells.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present invention. Thus, the appearances of the foregoing phrases in various places throughout this specification are not necessarily all referring to the same embodiment. Furthermore, the particular features, structures, or characteristics may be combined in any suitable manner in one or more embodiments.

“Treating” or treatment of a disease or condition refers to executing a protocol, which may include administering one or more drugs to a patient, in an effort to alleviate signs or symptoms of the disease. Desirable effects of treatment include decreasing the rate of disease progression, ameliorating or palliating the disease state, and remission or improved prognosis. Alleviation can occur prior to signs or symptoms of the disease or condition appearing, as well as after their appearance. Thus, “treating” or “treatment” may include “preventing” or “prevention” of disease or undesirable condition. In addition, “treating” or “treatment” does not require complete alleviation of signs or symptoms, does not require a cure, and specifically includes protocols that have only a marginal effect on the patient.

The term “therapeutic benefit” or “therapeutically effective” as used throughout this application refers to anything that promotes or enhances the well-being of the subject with respect to the medical treatment of this condition. This includes, but is not limited to, a reduction in the frequency or severity of the signs or symptoms of a disease. For example, treatment of cancer may involve, for example, a reduction in the size of a tumor, a reduction in the invasiveness of a tumor, reduction in the growth rate of the cancer, or prevention of metastasis. Treatment of cancer may also refer to prolonging survival of a subject with cancer.

“Subject” and “patient” and “individual” may be interchangeable and may refer to either a human or non-human, such as primates, mammals, and vertebrates. In particular embodiments, the subject is a human. The subject can be any organism or animal subject that is an object of a method or material, including mammals, e.g., humans, laboratory animals (e.g., primates, rats, mice, rabbits), livestock (e.g., cows, sheep, goats, pigs, turkeys, and chickens), household pets (e.g., dogs, cats, and rodents), horses, and transgenic non-human animals. The subject can be a patient, e.g., have or be suspected of having a disease (that may be referred to as a medical condition), such as one or more infectious diseases, one or more genetic disorders, one or more cancers, or any combination thereof. The “subject” or “individual”, as used herein, may or may not be housed in a medical facility and may be treated as an outpatient of a medical facility. The individual may be receiving one or more medical compositions via the internet. An individual may comprise any age of a human or non-human animal and therefore includes both adult and juveniles (e.g., children) and infants and includes in utero individuals. A subject may or may not have a need for medical treatment; an individual may voluntarily or involuntarily be part of experimentation whether clinical or in support of basic science studies.

The phrases “pharmaceutical or pharmacologically acceptable” refers to molecular entities and compositions that do not produce an adverse, allergic, or other untoward reaction when administered to an animal, such as a human, as appropriate. The preparation of a pharmaceutical composition comprising an antibody or additional active ingredient will be known to those of skill in the art in light of the present disclosure. Moreover, for animal (e.g., human) administration, it will be understood that preparations should meet sterility, pyrogenicity, general safety, and purity standards as required by FDA Office of Biological Standards.

The term “optionally” as used herein refers to an element, step, or parameter that may or may not be utilized in any method of the disclosure.

As used herein, “pharmaceutically acceptable carrier” includes any and all aqueous solvents (e.g., water, alcoholic/aqueous solutions, saline solutions, parenteral vehicles, such as sodium chloride, Ringer's dextrose, etc.), non-aqueous solvents (e.g., propylene glycol, polyethylene glycol, vegetable oil, and injectable organic esters, such as ethyloleate), dispersion media, coatings, surfactants, antioxidants, preservatives (e.g., antibacterial or antifungal agents, anti-oxidants, chelating agents, and inert gases), isotonic agents, absorption delaying agents, salts, drugs, drug stabilizers, gels, binders, excipients, disintegration agents, lubricants, sweetening agents, flavoring agents, dyes, fluid and nutrient replenishers, such like materials and combinations thereof, as would be known to one of ordinary skill in the art. The pH and exact concentration of the various components in a pharmaceutical composition are adjusted according to well-known parameters.

The term “viability” as used herein refers to the ability of a specific cell or plurality of cells to maintain a state of survival.

### II. Embodiments of the Methods

Embodiments of the disclosure include methods for identifying predictors for a response of immune cells, such as NK cells, derived from cord blood cells. In particular embodiments, cord blood units are tested for one or a variety of predictors that may produce immune cells better suited for adoptive cell therapy than cord blood units lacking in one or more of the predictors. Parameters being tested that can predict for an improved response of immune cells derived from cord blood cells in comparison to cells not so tested may comprise cell production, cell engineering, and/or cell activity processes. The parameters may regard the cord blood units themselves, or the parameters may regard any cells derived from the cord blood units, or manipulation or modification thereof. Such parameters include viability of cord blood units; red blood cell content of the cord blood units; total mononuclear cell recovery from the cord blood units; expansion of immune cells derived from thawed cord blood units (including at one or more ranges of time points); volumes of materials; gender, age and/or weight of the baby, race of one or more biological parents of the baby; one or more marker of the cells; engineering of immune cells derived from thawed cord blood units; cytotoxicity of immune cells derived from the thawed cord blood units; gestational age of a mother from which the cord blood is derived; cytotoxicity of immune cells derived from the thawed cord blood units (including cytotoxicity against cancer cells or cells infected with a pathogen); viability of cord blood units following thawing; and so forth.

Embodiments of the disclosure include methods for selecting cryopreserved cord blood units for the manufacture of cells for adoptive cell therapy having a higher potency (such as by being measured using cytotoxicity assays and the proportion of patients who respond) for a specific purpose, including clinical applications, than cells not so selected. In specific embodiments, the methods are for selecting cryopreserved cord blood units for the manufacture of engineered immune cells with a higher potency for adoptive cell therapy than cells not so selected, including for the treatment of cancer, for example. In particular aspects, the methods are for selecting cryopreserved cord blood units for the manufacture of engineered natural killer cells with a higher potency for adoptive cell therapy than cells not so selected, including for the treatment of cancer of any kind, for example.

In particular embodiments, methods encompassed herein include those in which a risk is reduced of selecting cord blood units (which may be referred to as cord blood compositions) that would produce immune cells, such as NK cells, that are ineffective or inferior at being engineered, expanded, and/or at being utilized clinically, such as for the treatment of cancer. In specific embodiments, the methods reduce the risk of selecting cord blood units that would produce immune cells lacking high potency, such as for cancer therapy as adoptive cell therapy. In specific cases, the methods encompassed herein increase the likelihood of producing adoptive NK cell therapy that is efficacious against one or more types of cancer.

The methods of the disclosure select for cells for adoptive cell therapy that are quantitatively and/or qualitatively better at cell therapy than cells not so selected. Qualitatively, the cells may be more cytotoxic, may expand to a greater capacity, may have greater persistence, may be more conducive to engineering, may have a greater proportion of patients who respond, or a combination thereof. Quantitatively, the selected cord blood units from the method may have cell viability levels that are at least at least 10%, 15%, 20%, 25%, 30%, 35%, 40%, 45%, 50%, 55%, 65%, 70%, 75%, 80%, 85%, 90%, 95%, 96%, 97%, 98%, 99%, or greater compared to cord blood units selected without knowledge of one or more of the selection parameters encompassed herein. The selected cord blood units from the method may have cell viability levels that are at least at least 10-fold, 20-fold, 30-fold, 40-fold, 50-fold, 60-fold, 70-fold, 80-fold, 90-fold, 100-fold, 250-fold, 500-fold, 750-fold, 1000-fold, or greater compared to cord blood units selected without knowledge of one or more of the selection parameters encompassed herein. The selected cord blood units from the method may produce total mononuclear cell recovery that is greater than at least 10%, 15%, 20%, 25%, 30%, 35%, 40%, 45%, 50%, 55%, 65%, 70%, 75%, 80%, 85%, 90%, 95% or more than cord blood units selected without knowledge of one or more of the selection parameters encompassed herein. The selected cord blood units from the method may produce total mononuclear cell recovery that is greater than at least 10-fold, 20-fold, 30-fold, 40-fold, 50-fold, 60-fold, 70-fold, 80-fold, 90-fold, 100-fold, 250-fold, 500-fold, 750-fold, 1000-fold, or greater or more than cord blood units selected without knowledge of one or more of the selection parameters encompassed herein. The selected cord blood units from the method may have a nucleated red blood cell content that is at least 1×10³, 1×10⁴, 1×10⁵, 1×10⁶, 1×10⁷, 5×10⁷, or lower than cord blood units selected without knowledge of one or more of the selection parameters encompassed herein. The selected cord blood units from the method may have a nucleated red blood cell content that is at least 10%, 20%, 30%, 40%, 50%, 60%, 70%, 80%, 90%, or lower than cord blood units selected without knowledge of one or more of the selection parameters encompassed herein.

In certain embodiments, the weight of the baby at the time of collection of cord blood tissue may be considered in methods of the disclosure, whether or not in utero or ex utero. In specific embodiments, the weight of the baby is greater than 3650 grams, such as greater than 3650, 3700, 3750, 3800, 3850, 3900, 3950, 4000, 4050, 4100, 4150, 4200, 4250, 4500 grams, and so forth. In specific embodiments, this is measured prior to cryopreservation and/or use.

In specific embodiments, the race of one or more biological parents of the baby is Caucasian. In some cases, both biological parents of the baby are Caucasian, in some cases the biological mother is Caucasian, and in some cases the biological father is Caucasian.

In specific embodiments, the timing of collection of the cord blood from the baby is a factor in the method. In specific embodiments, the cord blood is obtained from the cord of the baby in utero. The collection step may be by any suitable method, and the party obtaining the cord blood may or may not be the party that manipulates, stores, and/or analyzes the cord blood for one or more parameters. In specific embodiments, upon collection or soon thereafter the cord blood is combined with one or more anticoagulants and the volume of the anticoagulant may or may not be a standard amount. In specific cases, the preprocess volume is the volume of cord blood collected plus anticoagulant, and in certain cases the preprocess volume is the volume of cord blood collected plus anticoagulant of a specific volume, such as 35 mL or about 35 mL. In particular embodiments, the volume of the extracted cord blood is no greater than about 85, 84, 83, 82, 81, 80, 79, 78, 77, 76, 75, 74, 73, 72, 71, 70, 69, 68, 67, 66, 65, 64, 63, 62, 61, 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, or 30 mL or less in volume. In some cases, the volume of the anticoagulant is or is about 25, 26, 27, 28, 29, 30, 31, 32, 33, 34, 35, 36, 37, 38, 39, 40, 41, 42, 43, 44, 45, 46, 47, 48, 49, or 50 mL or more. In specific cases, the volume of the anticoagulant is or is about 35 mL. The anticoagulant may be of any kind, including at least CPD (and may be CDP-A (CDP+adenosine); citrate-phosphate-soluble dextrose (CP2D); acid citrate dextrose (ACD); Heparin, etc.). In particular embodiments, cells in the collected cord blood may express one or more particular markers. In specific cases, cells in the collected cord blood may express CD34. In certain embodiments, a particular percentage of cells express any marker, including CD34. In certain cases, >0.4% cells in the collected blood express CD34. In certain cases, >0.4, 0.5, 0.6, 0.7, 0.8, 0.9, 1.0, 1.5, 2, 3, 4, 5, 6, 7, 8, 9, 10, 15, 20, 25, 30, 40, 50, 60, 70, 75, 80, 85, 90, or 95% cells in the collected blood express CD34. In certain cases, at least 0.4, 0.5, 0.6, 0.7, 0.8, 0.9, 1.0, 1.5, 2, 3, 4, 5, 6, 7, 8, 9, 10, 15, 20, 25, 30, 40, 50, 60, 70, 75, 80, 85, 90, or 95% cells in the collected blood express CD34. In specific embodiments, this is measured prior to cryopreservation and/or use.

The selected cord blood cells from the method may produce immune cells that have cytotoxicity levels that are at least 10%, 15%, 20%, 25%, 30%, 35%, 40%, 45%, 50%, 55%, 65%, 70%, 75%, 80%, 85%, 90%, 95%, or greater compared to immune cells produced from cord blood cells selected without knowledge of one or more of the selection parameters encompassed herein. In some cases, the immune cells produced from the selected cord blood cells may have cytotoxicity levels that are at least 10-fold, 20-fold, 30-fold, 40-fold, 50-fold, 60-fold, 70-fold, 80-fold, 90-fold, 100-fold, 250-fold, 500-fold, 750-fold, 1000-fold, or greater compared to immune cells selected from cord blood cells without knowledge of one or more of the selection parameters encompassed herein.

Particular aspects of the disclosure select for one or more product characteristics of cord blood units prior to freezing of any kind, and in some aspects there are one or more product characteristics selected for following thawing of the frozen cord blood units. Such action(s) allows for selecting cord blood units that are best suited (among a collection of cord blood units from which to choose) to produce cell products, including cell products for adoptive cell therapy. The characteristics of cord blood units post-thaw may or may not be directly related to production of the cell product. That is, in some cases, the production of cell therapy by engineering of the cells derived from the cord blood units is enhanced by selecting the appropriate cord blood units, and in additional or alternative cases, the activity of cell therapy following engineering of cells derived from the cord blood units is enhanced by selecting the appropriate cord blood units (e.g., activity such as cytoxicity, persistence in vivo, and so forth).

Embodiments of the disclosure include methods in which one or more parameters are characterized for one or more cord blood units from one or more storage banks of any kind of cord blood units. In specific cases, following characterization of the one or more cord blood units, one or more particular cord blood units may be rejected as being unsuitable to provide for optimal responses (e.g., activity upon therapeutic administration). In additional cases, one or more particular cord blood units may be determined to be suitable for enhanced activities, such as upon therapeutic administration. In some situations when more than one cord blood unit is determined by methods of the disclosure to be worthy of selection, they may or may not be combined prior to thawing or subsequent to thawing. Immune cells produced from selected cord blood units may be combined following derivation from the cord blood units.

In specific embodiments, the disclosure provides a novel set of criteria to identify cord blood units for the manufacture of NK cell therapy products with the highest potency for the treatment of cancer. NK cells generated from these highly potent cord blood units are most likely to result in an optimal response in cancer patients. As such, the methods of the disclosure are used to select cord blood units with the highest potency as a material source for the manufacture of NK cell therapy products and to avoid the selection of cord blood units and/or the generation of NK cells unlikely to induce a clinical response or likely to induce an ineffective clinical response. In particular embodiments, high potency NK cells produced from cord blood units selected by methods of the disclosure have the highest probability of inducing remissions in patients with cancer following adoptive infusion. In specific cases, high potency NK cells produced from cord blood units selected by methods of the disclosure have a greater probability of inducing remissions in patients with cancer following adoptive infusion than NK cells produced from cord blood units that lack the disclosed beneficial characteristics.

Embodiments of the disclosure include methods of selecting a cord blood composition, comprising the steps of identifying a cord blood composition that, prior to cryopreservation, is determined to have one or more of the following: (a) cord blood cell viability greater than or equal to 98% or 99%; (b) optionally total mononuclear cell (TNC) recovery is greater than or equal to 76.3%; and (c) nucleated red blood cell (NRBC) content less than or equal to 7.5×10⁷-8.0×10⁷ and any amount therebetween; (d) weight of the baby from which the cord blood is derived; (e) race of the biological mother and/or biological father of the baby from which the cord blood is derived is Caucasian; (f) optionally gestational age of the baby from which the cord blood is derived; (g) optionally intra utero collection of the cord blood (although extra utero or a combination of intra utero and extra utero may be used in any method of the disclosure); (h) optionally a biologically male baby from which the cord blood is derived; (i) optionally a pre-process volume (volume of the cord blood collected plus anticoagulant (35 ml CPD)) ≤120 mL; (j) optionally, cells of the extracted cord blood are >0.4% CD34+ and optionally (k) measuring cytotoxicity of immune cells derived from the cord blood composition following thawing; and optionally (1) measuring expansion of the cells in culture. In some cases, the cord blood composition prior to cryopreservation is determined to have at least the characteristics of (a) and (c). In some cases, the cord blood composition prior to cryopreservation is determined to have at least the characteristics of (b) and (c). In some cases, the cord blood composition prior to cryopreservation is determined to have at least the characteristics of (a) and (b). In some cases, the cord blood composition prior to cryopreservation is determined to have 1, 2, 3, or all of the characteristics of (a), (c), (d), and (e), and they may be in any combination. In some cases, the cord blood composition prior to cryopreservation is determined to have (a), (b), (c), and (d). In some cases, the cord blood composition prior to cryopreservation is determined to have 1, 2, 3, or all of the characteristics of (a), (c), (d), and (e) in addition to one or more of (b) (f), (g), (h), (i), and (j).

A. Measurement of Cell Viability

Embodiments of the disclosure include methods in which the viability of cells in cord blood units is measured, and the measurement provides information whether or not the cord blood unit is suitable, such as suitable for selection for derivation of immune cells for adoptive cell therapy. The cord blood cells being tested for viability may be a mixture of cells in the cord blood, such as mononuclear, stem cells (e.g., hematopoietic or mesenchymal), white cells, immune system cells (monocytes, macrophages, neutrophils, basophils, eosinophils, megakaryocytes, dendritic cells, T cells (including T helper and cytotoxic), B cells, NK cells), and so forth. The viability of cells in the cord blood can be observed through one or more physical properties of the cells and/or one or more activities of the cells.

Although the viability may be determined by any suitable method(s), in specific cases the measurements are performed by flow cytometry, tetrazolium reduction assay, resazurin reduction assay, protease viability marker assay, ATP Assay, sodium-potassium ratio, lactate dehydrogenase assay, neutral red uptake, propidium iodide, TUNEL assay, formazan-based assay, Evans blue, Trypan blue, ethidium homodimer assay, or a combination thereof.

Cell viability for cord blood cells may be measured prior to cryopreservation and/or subsequent to cryopreservation. In one embodiment, cord blood cell viability for a desired cord blood unit is greater than or equal to 98.1, 98.2, 98.3, 98.4, 98.5, 98.6, 98.7, 98.8, 98.9, 99.0, 99.1, 99.2, 99.3, 99.4, 99.5, 99.6, 99.7, 99.8, or 99.9%.

In cases wherein viability is measured in addition to one or more other characteristics, such as total nuclear cell recovery and measurement of nucleated red blood cell content, the cell viability may or may not be prior to one or more other measurements. In specific cases, viability is measured prior to TNC recovery and NRBC measurement or is measured subsequent to TNC recovery and NRBC measurement. In some cases, viability is measured after TNC but before NRBC or is measured after NRBC but before TNC recovery.

B. Measurement of Total Nuclear Cell Recovery

In particular embodiments of the method, the total nuclear cell (TNC) recovery is measured in which nucleated cells are measured following cord blood processing. The TNC recovery measures nucleated cells that are both live and dead. This step may or may not be optional.

Any suitable assay for measurement of TNC may be utilized, but in specific embodiments, the TNC recovery assay includes flow cytometry; Trypan blue; 3% Acetic Acid with Methylene Blue; hematology analyzer analysis; or a combination thereof. The TNC recovery assay may or may not be automated, in specific cases.

In one embodiment, TNC recovery is greater than or equal to 76.3, 76.4, 76.5, 76.6, 76.7, 76.8, 76.9, 77, 78, 79, 80, 81, 82, 83, 84, 85, 86, 87, 88, 89, 90, 91, 92, 93, 94, 95, 96, 97, 98, or 99%.

In particular embodiments, TNC recovery of cord blood units is measured prior to cryopreservation.

In cases wherein TNC recovery is measured in addition to one or more other characteristics, such as cell viability and measurement of NRBC content, the TNC recovery may or may not be prior to one or more other measurements. In specific cases, TNC recovery is measured prior to cell viability and NRBC measurement or is measured subsequent to cell viability and NRBC measurement. In some cases, TNC recovery is measured after cell viability but before NRBC or is measured after NRBC but before cell viability.

C. Measurement of Nucleated Red Blood Count

In particular embodiments, cord blood units are selected based on the measurement of nucleated red blood cell (NRBC) content. The measurement may be manual or automated. In particular embodiments, cord blood units with lower NRBC content are more effective at producing efficacious immune cells than cord blood units with higher NRBC content. The level of NRBC in cord blood units determines the response rate of individuals treated with immune cells, such as NK cells, derived from the particular cord blood unit. The NRBC content may be measured by density centrifugation, such as on a Sepax® device.

In specific embodiments, the NRBC content is less than or equal to 8.0×10⁷, 7.9×10⁷, 7.8×10⁷, 7.7×10⁷, 7.6×10⁷, 7.5×10⁷, 7.0×10⁷, 6.0×10⁷, 5.0×10⁷, 4.0×10⁷, 3.0 ×10⁷, 2.0×10⁷, 1.0×10⁷, 9.0×10⁶, 8.0×10⁶, 7.0×10⁶, 6.0×10⁶, 5.0×10⁶, 4.0×10⁶, 3.0×10⁶, 2.0×10⁶, 1.0×10⁶, 9.0×10⁵, 8.0×10⁵, 7.0×10⁵, 6.0×10⁵, 5.0×10⁵, 4.0×10⁵, 3.0×10⁵, 2.0 ×10⁵, 1.0×10⁵, 9.0×10⁴, 8.0×10⁴, 7.0×10⁴, 6.0×10⁴, 5.0×10⁴, 4.0×10⁴, 3.0×10⁴, 2.0×10⁴, 1.0×10⁴, 9.0×10³, 8.0×10³, 7.0×10³, 6.0×10³, 5.0×10³, 4.0×10³, 3.0×10³, 2.0×10³, 1.0×10¹, 9.0×10², 8.0×10², 7.0×10², 6.0×10², 5.0×10², 4.0×10², 3.0×10², 2.0×10², 1.0×10², and so forth, including to an undetectable level.

In particular embodiments, NRBC is measured prior to cryopreservation.

In cases wherein NRBC is measured in addition to one or more other characteristics, such as total nuclear cell recovery and cell viability, the NRBC may or may not be prior to one or more other measurements. In specific cases, NRBC is measured prior to TNC recovery and cell viability or is measured subsequent to TNC recovery and cell viability. In some cases, NRBC is measured after TNC but before cell viability or is measured after cell viability but before TNC recovery.

D. Weight of the Baby

In some embodiments, the weight of the baby from which the cord blood is derived is utilized as a parameter in any method encompassed by the disclosure. The weight of the baby may be taken just prior to collection of the cord blood, such as within days or hours or minutes, for example. In some cases, the weight of the baby may be determined in utero by using prenatal ultrasound. In some cases, the weight of the baby is determined ex utero, such as on a standard scale. The party measuring the weight of the baby may or may not be the party that manipulates, stores, and/or analyzes the cord blood for one or more parameters. This step may occur before and/or after any other step prior to cryopreservation. In specific embodiments, the weight of the baby is greater than a certain amount, and this may or may not generally correlated with gestational age. In specific cases, the weight of the baby is greater than about 3650 grams, such as greater than 3650, 3700, 3750, 3800, 3850, 3900, 3950, 4000, 4050, 4100, 4150, 4200, 4250, 4500 grams, and so forth. In specific embodiments, this is measured prior to cryopreservation and/or use.

E. Race of the Biological Parents

In specific embodiments, the race of one or more of the biological parents is Caucasian. In some cases the biological mother is Caucasian and the biological father is Caucasian. In some cases, the biological mother is Caucasian but the biological father is not Caucasian. In some cases, the biological father is Caucasian but the biological mother is not Caucasian.

F. Collection Parameters for Cord Blood

In some embodiments, the cord blood is obtained by standard methods in the art, such as via a needle from the umbilical vein after the baby is born. For ex utero extraction, this is done after the placenta has been expelled, and the cord blood is inserted into a sterile collection bag that comprises an anticoagulant, or an anticoagulant may be added. For in utero extraction, this is done through the umbilical vein while the placenta is still inside the mother, following which it is inserted into a sterile collection bag that comprises an anticoagulant, or an anticoagulant may be added. In some cases, cord blood from the same baby is combined from in utero and ex utero extractions. In a specific embodiment, in utero extraction is a method of choice over ex utero extraction.

In particular embodiments, the volume of extracted cord blood is considered in the methods of the disclosure. For example, the volume of the combination of both cord blood and anticoagulant as a pre-processing composition is considered in methods of the disclosure. In specific cases, the volume of the combination of cord blood and anticoagulant is ≤120 mL. As one example, when the volume of anticoagulant is about 35 mL, the volume of the cord blood is less than about 85, 84, 83, 82, 81, 80, 79, 78, 77, 76, 75, 74, 73, 72, 71, 70, 69, 68, 67, 66, 65, 64, 63, 62, 61, 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, or 30 mL in volume.

G. Cord Blood Cell Markers

In specific embodiments, at least some of cells of any type in the cord blood may collectively express one or more particular markers. In specific embodiments, a particular percentage of cells of the cord blood express CD34. Examples of cord blood cell types include stem cells, progenitor cells, red blood cells, white blood cells, B lymphocytes, T lymphocytes, NK cells, monocytes, and platelets. In some cases, >0.4% cells in the collected cord blood express CD34. In certain cases, >0.4, 0.5, 0.6, 0.7, 0.8, 0.9, 1.0, 1.5, 2, 3, 4, 5, 6, 7, 8, 9, 10, 15, 20, 25, 30, 40, 50, 60, 70, 75, 80, 85, 90, or 95% cells in the collected blood express CD34. In certain cases, at least 0.4, 0.5, 0.6, 0.7, 0.8, 0.9, 1.0, 1.5, 2, 3, 4, 5, 6, 7, 8, 9, 10, 15, 20, 25, 30, 40, 50, 60, 70, 75, 80, 85, 90, or 95% cells in the collected blood express CD34. In specific embodiments, this is measured prior to cryopreservation and/or use.

H. Measurement of Cytotoxicity

Embodiments of the disclosure include measurement of cytotoxicity of immune cells of any kind, including NK cells, derived from cord blood units. In specific embodiments, there is measurement of cytotoxicity of NK cells derived from the cord blood unit(s). In particular cases, cord blood cell unit(s) are characterized for viability, NRBC, and TNC recovery, and following the selection of the cord blood cell unit(s) based on this characterization, and optionally following cryopreservation and thawing, cells from the cord blood unit(s) may be measured for cytotoxicity.

Cytotoxicity assays often rely on dying cells having highly compromised cellular membranes that allow the release of cytoplasmic content or the penetration of fluorescent dyes within the cell structure. Cytotoxicity can be measured in a number of different ways, such as measuring cell viability using vital dyes (formazan dyes), protease biomarkers, or by measuring ATP content, for example. The formazan dyes are chromogenic products formed by the reduction of tetrazolium salts (INT, MTT, MTS and XTT) by dehydrogenases, such as lactate dehydrogenase (LDH) and reductases that are released at cell death. Other assays include sulforhodamine B and water-soluble tetrazolium salt assays that may be utilized for high throughput screening. One can measure cytotoxicity with an Incucyte® device.

In specific embodiments, one can utilize dyes that selectively penetrate dead cells, such as Trypan blue. In other cases, one can utilize fluorescent DNA binding dyes that penetrate dead cells, such as Hoechst 33342, YO-PRO-1, or CellTox Green.

In specific embodiments wherein the cells being tested for being cytotoxic are T cells or NK cells, one may utilize the ⁵¹Cr release assay.

I. Measurement of NK Cell Expansion

In specific embodiments of methods of the disclosure, the extent of NK cell expansion following cryopreservation and thawing of cord blood units (including cord blood units selected based on criteria encompassed herein) is a predictor of clinical response. That is, following thawing of cord blood, the thawed blood is processed and cultured under conditions such that the quantity of NK cells in the culture is increased. Cord blood units that meet selection criteria encompassed herein may or may not be pooled prior to expansion of NK cells. The quantitative extent of the NK cell expansion, including at certain time points in some cases, in some embodiments is utilized as a selection criteria for NK cells that will have greater clinical efficacy compared to NK cells derived from randomly selected cord blood units.

In particular cases, the NK cells are expanded, and the expansion level is determined. When the NK cells at a certain time point are expanded to at least a particular level, the NK cells have a greater clinical efficacy compared to NK cells that are not able to be expanded to such a level. In at least some cases, NK cells that would have clinical efficacy at a range between days 0 and 6 is greater than or equal to 2-fold, 3-fold, 4-fold, 5-fold, 6-fold, 7-fold (including 8-fold, 9-fold, 10-fold, 12-fold, 15-fold, 20-fold, 50-fold, 100-fold, 150-fold, 200-fold, 250-fold, 500-fold, 1000-fold, 1500-fold, 2000-fold, and so forth). In at least some cases, NK cells may have an insufficient clinical efficacy if at a range between days 0 and 6 the expansion is less than 7-fold (including less than 6-fold, 5-fold, 4-fold, 3-fold, or 2-fold). In at least some cases, NK cells would have clinical efficacy if at a range between days 6 and 15 the expansion in culture is greater than or equal to 10²-fold, 10³-fold, 10⁴-fold, 10⁵-fold (including 10⁶-fold, 10⁷-fold, 10⁸-fold, 10⁹-fold, 10¹⁰-fold, 10¹¹-fold, 10¹²-fold, 10¹³-fold, and so forth). In at least some cases, NK cells may have an insufficient clinical efficacy if at a range between days 6 and 15 the expansion in culture is less than 10⁵-fold (including less than 10⁴-fold, 10³-fold, 10²-fold, and so forth). In at least some cases, NK cells that would have clinical efficacy at a range between days 0 and 15 is greater than or equal to 900-fold, 1000-fold, 1100-fold, 1200-fold, 1300-fold, 1400-fold, 1500-fold, 1600-fold, 1700-fold, 1800-fold, 1900-fold, 2000-fold, 2500-fold, 3000-fold, 4000-fold, 5000-fold, 10,000-fold, or greater. In at least some cases, NK cells may have an insufficient clinical efficacy if at a range between days 6 and 15 the expansion in culture is less than 900-fold, such as less than 800-fold, 700-fold, 600-fold, 500-fold, 400-fold, 300-fold, 200-fold, 100-fold, and so forth.

In some embodiments, the NK cell expansion utilizes a particular in vitro method for expanding NK cells. In some cases, there is pre-activation of a population of NK cells in a pre-activation culture comprising an effective concentration of IL-12, IL-15, and/or IL-18 to obtain pre-activated NK cells; and then expanding the pre-activated NK cells in an expansion culture comprising artificial antigen presenting cells (aAPCs) expressing CD137 ligand. In certain aspects, the aAPCs further express a membrane-bound cytokine. In some aspects, the membrane-bound cytokine is membrane-bound IL-21 (mIL-21) and/or membrane-bound IL-15 (mIL-15). In some aspects, the aAPCs have essentially no expression of endogenous HLA class I, II, or CD1d molecules. In certain aspects, the aAPCs express ICAM-1 (CD54) and LFA-3 (CD58). In some aspects, the aAPCs are further defined as leukemia cell-derived aAPCs. In certain aspects, the leukemia-cell derived aAPCs are further defined as K562 cells engineered to express CD137 ligand and/or mIL-21. The K562 cells may be engineered to express CD137 ligand and mIL-21. In certain aspects, engineered is further defined as retroviral transduction. In particular aspects, the aAPCs are irradiated. In particular cases, the pre-activating step is for 10-20 hours, such as 14-18 hours (e.g., about 14, 15, 16, 17, or 18 hours), particularly about 16 hours. In certain aspects, the pre-activation culture comprises IL-18 and/or IL-15 at a concentration of 10-100 ng/mL, such as 40-60 ng/mL, particularly about 50 ng/mL. In some aspects, the pre-activation culture comprises IL-12 at a concentration of 0.1-150 ng/mL, such as 1-20 ng/mL, particularly about 10 ng/mL. In additional aspects, the expansion culture further comprises IL-2. In some aspects, the IL-2 is present at a concentration of 10-500 U/mL, such as 100-300 U/mL, particularly about 200 U/mL. In some aspects, the IL-12, IL-18, IL-15, and/or IL-2 is recombinant human IL-2. In some aspects, the IL-2 is replenished in the expansion culture every 2-3 days. In some aspects, the aAPCs are added to the expansion culture at least a second time. In some aspects, the method is performed in serum-free media.

In one embodiment, the expansion step comprises culturing the NK cells in the presence of an effective amount of universal antigen presenting cells (UAPC) engineered to express (1) CD48 and/or CS1 (CD319), (2) membrane-bound interleukin-21 (mbIL-21), and (3) 41BB ligand (41BBL)). In some aspects, the immune cells and UAPCs are cultured at a ratio of 3:1 to 1:3, such as 3:1, 3:2, 1:1, 1:2, or 1:3. In particular aspects, the immune cells and UAPCs are cultured at a ratio of 1:2. In some aspects, the UAPC has essentially no expression of endogenous HLA class I, II, or CD1d molecules. In certain aspects, the UAPC expresses ICAM-1 (CD54) and LFA-3 (CD58). In certain aspects, the UAPC is further defined as a leukemia cell-derived aAPC. In some aspects, the leukemia-cell derived UAPC is further defined as a K562 cell. In certain aspects, the UAPCs are added at least a second time.

In some aspects, the expanding is in the presence of IL-2. In specific aspects, the IL-2 is present at a concentration of 10-500 U/mL, such as 10-25, 25-50, 50-75, 75-10, 100-150, 150-200, 200-250, 250-300, 300-350, 350-400, or 400-500 U/mL. In certain aspects, the IL-2 is present at a concentration of 100-300 U/mL. In particular aspects, the IL-2 is present at a concentration of 200 U/mL. In some aspects, the IL-2 is recombinant human IL-2. In specific aspects, the IL-2 is replenished every 2-3 days, such as every 2 days or 3 days.

### III. Immune Cells Derived from the Cord Blood

Certain embodiments of the present disclosure concern immune cells that are derived from cord blood unit(s) that are selected for processing based upon one or more criteria encompassed herein. The immune cells may be of any kind including NK cells, invariant NK cells, NKT cells, T cells (e.g., regulatory T cells, CD4⁺ T cells, CD8⁺ T cells, or gamma-delta T cells), monocytes granulocytes, myeloid cells, macrophages, neutrophils, dendritic cells, mast cells, eosinophils, basophils, stem cells (e.g., mesenchymal stem cells (MSCs) or induced pluripotent stem (iPSC) cells), and so forth. Also provided herein are methods of producing and engineering the immune cells as well as methods of using and administering the cells for adoptive cell therapy, in which case the cells may be autologous or allogeneic with respect to the individual(s) from which the cord blood was obtained. Thus, the immune cells may be used as immunotherapy, such as to target cancer cells.

The immune cells may be isolated from cord blood units from human subjects. The cord blood can be obtained from a subject of interest, such as a subject suspected of having a particular disease or condition, a subject suspected of having a predisposition to a particular disease or condition, or a subject who is undergoing therapy for a particular disease or condition. The cord blood can be obtained from a subject for the purpose of banking the cord blood in case it (including immune cells derived from it) is needed later in lift. The immune cells derived from the cord blood may be used directly, or they can be stored for a period of time, such as by freezing. The cord blood may or may not be pooled, such as may be from 2 or more sources, such as 3, 4, 5, 6, 7, 8, 9, 10 or more sources (e.g., donor subjects).

The cord blood from which the immune cells are derived can be obtained from a subject in need of therapy or suffering from a disease of any kind, including associated with reduced immune cell activity. Thus, the cells will be autologous to the subject in need of therapy. Alternatively, the population of immune cells can be obtained from a donor, preferably a histocompatibility matched donor. The immune cell population can be harvested from the peripheral blood, cord blood, bone marrow, spleen, or any other organ/tissue in which immune cells reside in said subject or donor. The immune cells can be isolated from a pool of subjects and/or donors, such as from pooled cord blood.

When the population of immune cells is obtained from cord blood units from a donor distinct from the subject, the donor is preferably allogeneic, provided the cells obtained are subject-compatible in that they can be introduced into the subject. Allogeneic donor cells are may or may not be human-leukocyte-antigen (HLA)-compatible. To be rendered subject-compatible, allogeneic cells can be treated to reduce immunogenicity.

A. NK Cells

In some embodiments, the immune cells derived from the selected cord blood unit(s) are NK cells. NK cells are a subpopulation of lymphocytes that have spontaneous cytotoxicity against a variety of tumor cells, virus-infected cells, and some normal cells in the bone marrow and thymus. NK cells are critical effectors of the early innate immune response toward transformed and virus-infected cells. NK cells constitute about 10% of the lymphocytes in human peripheral blood. When lymphocytes are cultured in the presence of IL-2, strong cytotoxic reactivity develops. NK cells are effector cells known as large granular lymphocytes because of their larger size and the presence of characteristic azurophilic granules in their cytoplasm. NK cells differentiate and mature in the bone marrow, lymph nodes, spleen, tonsils, and thymus. NK cells can be detected by specific surface markers, such as CD16, CD56, and CD8 in humans. NK cells do not express T cell antigen receptors, the pan T marker CD3, or surface immunoglobulin B cell receptors.

Stimulation of NK cells is achieved through a cross-talk of signals derived from cell surface activating and inhibitory receptors. The activation status of NK cells is regulated by a balance of intracellular signals received from an array of germ-line-encoded activating and inhibitory receptors (Campbell, 2006). When NK cells encounter an abnormal cell (e.g., tumor or virus-infected cell) and activating signals predominate, the NK cells can rapidly induce apoptosis of the target cell through directed secretion of cytolytic granules containing perforin and granzymes or engagement of death domain-containing receptors. Activated NK cells can also secrete type I cytokines, such as interferon-.gamma., tumor necrosis factor-.alpha. and granulocyte-macrophage colony-stimulating factor (GM-CSF), which activate both innate and adaptive immune cells as well as other cytokines and chemokines (Wu et al., 2003). Production of these soluble factors by NK cells in early innate immune responses significantly influences the recruitment and function of other hematopoietic cells. Also, through physical contacts and production of cytokines, NK cells are central players in a regulatory crosstalk network with dendritic cells and neutrophils to promote or restrain immune responses.

In certain aspects, the NK cells are isolated and expanded by the previously described method of ex vivo expansion of NK cells (Shah et al., 2013). In this method, CB mononuclear cells are isolated by Ficoll density gradient centrifugation and cultured in a bioreactor with IL-2 and artificial antigen presenting cells (aAPCs). After 7 days, the cell culture is depleted of any cells expressing CD3 and re-cultured for an additional 7 days. The cells are again CD3-depleted and characterized to determine the percentage of CD56⁺/CD3⁻ cells or NK cells. In other methods, umbilical CB is used to derive NK cells by the isolation of CD34⁺ cells and differentiation into CD56⁺/CD3⁻ cells by culturing in medium contain SCF, IL-7, IL-15, and IL-2.

B. T Cells

In some embodiments, the immune cells derived from the selected cord blood unit(s) are T cells. Several basic approaches for the derivation, activation and expansion of functional anti-tumor effector cells have been described in the last two decades. These include: autologous cells, such as tumor-infiltrating lymphocytes (TILs); T cells activated ex-vivo using autologous DCs, lymphocytes, artificial antigen-presenting cells (APCs) or beads coated with T cell ligands and activating antibodies, or cells isolated by virtue of capturing target cell membrane; allogeneic cells naturally expressing anti-host tumor TCR; and non-tumor-specific autologous or allogeneic cells genetically reprogrammed or “redirected” to express tumor-reactive TCR or chimeric TCR molecules displaying antibody-like tumor recognition capacity known as “T-bodies”. These approaches have given rise to numerous protocols for T cell preparation and immunization which can be used in the methods described herein.

In some embodiments, one or more subsets of T cells are derived from the selected cord blood, such as CD4⁺ cells, CD8⁺ cells, and subpopulations thereof, such as those defined by function, activation state, maturity, potential for differentiation, expansion, recirculation, localization, and/or persistence capacities, antigen-specificity, type of antigen receptor, presence in a particular organ or compartment, marker or cytokine secretion profile, and/or degree of differentiation. With reference to the subject to be treated, the T cells may come from cord blood that is allogeneic or autologous, or a mixture thereof.

Certain types of T cells may be derived from the selected cord blood. Among the sub-types and subpopulations of T cells (e.g., CD4.sup.+ and/or CD8.sup.+ T cells) there are naive T (T_{N}) cells, effector T cells (T_{EFF}), memory T cells and sub-types thereof, such as stem cell memory T (TSC_{M}), central memory T (TC_{M}), effector memory T (T_{EM}), or terminally differentiated effector memory T cells, tumor-infiltrating lymphocytes (TIL), immature T cells, mature T cells, helper T cells, cytotoxic T cells, mucosa-associated invariant T (MAIT) cells, naturally occurring and adaptive regulatory T (Treg) cells, helper T cells, such as TH1 cells, TH2 cells, TH3 cells, TH17 cells, TH9 cells, TH22 cells, follicular helper T cells, alpha/beta T cells, and delta/gamma T cells.

In some embodiments, one or more of the T cell populations derived from the cord blood is enriched for or depleted of cells that are positive for one or more specific markers, such as surface markers, or that are negative for one or more specific markers. In some cases, such markers are those that are absent or expressed at relatively low levels on certain populations of T cells (e.g., non-memory cells) but are present or expressed at relatively higher levels on certain other populations of T cells (e.g., memory cells).

In some embodiments, T cells are separated from the cord blood sample by negative selection of markers expressed on non-T cells, such as B cells, monocytes, or other white blood cells, such as CD14. In some aspects, a CD4⁺ or CD8⁺ selection step is used to separate CD4⁺ helper and CD8⁺ cytotoxic T cells. Such CD4⁺ and CD8⁺ populations can be further sorted into sub-populations by positive or negative selection for markers expressed or expressed to a relatively higher degree on one or more naive, memory, and/or effector T cell subpopulations.

In some embodiments, CD8⁺ T cells are further enriched for or depleted of naive, central memory, effector memory, and/or central memory stem cells, such as by positive or negative selection based on surface antigens associated with the respective subpopulation.

In some embodiments, the T cells are cultured in interleukin-2 (IL-2), and in any case they may be pooled prior to expansion. Expansion can be accomplished by any of a number of methods as are known in the art. For example, T cells can be rapidly expanded using non-specific T-cell receptor stimulation in the presence of feeder lymphocytes and either interleukin-2 (IL-2) or interleukin-15 (IL-15). The non-specific T-cell receptor stimulus can include around 30 ng/ml of OKT3, a mouse monoclonal anti-CD3 antibody (available from Ortho-McNeil®, Raritan, N.J.). Alternatively, T cells can be rapidly expanded by stimulation of peripheral blood mononuclear cells (PBMC) in vitro with one or more antigens (including antigenic portions thereof, such as epitope(s), or a cell) of the cancer, which can be optionally expressed from a vector, such as an human leukocyte antigen A2 (HLA-A2) binding peptide, in the presence of a T-cell growth factor, such as 300 IU/ml IL-2 or IL-15. The in vitro-induced T cells are rapidly expanded by re-stimulation with the same antigen(s) of the cancer pulsed onto HLA-A2-expressing antigen-presenting cells. Alternatively, the T-cells can be re-stimulated with irradiated, autologous lymphocytes or with irradiated HLA-A2+ allogeneic lymphocytes and IL-2, for example.

C. Stem Cells

In some embodiments, the immune cells derived from the selected cord blood unit(s) may be stem cells, such as induced pluripotent stem cells (PSCs), mesenchymal stem cells (MSCs), or hematopoietic stem cells (HSCs), or a mixture thereof.

The pluripotent stem cells encompassed herein may be induced pluripotent stem (iPS) cells, commonly abbreviated iPS cells or iPSCs. The induction of pluripotency was originally achieved in 2006 using mouse cells (Yamanaka et al. 2006) and in 2007 using human cells (Yu et al. 2007; Takahashi et al. 2007) by reprogramming of somatic cells via the introduction of transcription factors that are linked to pluripotency. The use of iPSCs circumvents most of the ethical and practical problems associated with large-scale clinical use of ES cells, and patients with iPSC-derived autologous transplants may not require lifelong immunosuppressive treatments to prevent graft rejection.

Somatic cells, such as those in the cord blood unit, can be reprogrammed to produce iPS cells using methods known to one of skill in the art. One of skill in the art can readily produce iPS cells, see for example, Published U.S. Patent Application No. 2009/0246875, Published U.S. Patent Application No. 2010/0210014; Published U.S. Patent Application No. 2012/0276636; U.S. Pat. Nos. 8,058,065; 8,129,187; PCT Publication NO. WO 2007/069666 A1, U.S. Pat. Nos. 8,268,620; 8,546,140; 9,175,268; 8,741,648; U.S. Patent Application No. 2011/0104125, and U.S. Pat. No. 8,691,574, which are incorporated herein by reference. Generally, nuclear reprogramming factors are used to produce pluripotent stem cells from a somatic cell. In some embodiments, at least three, or at least four, of Klf4, c-Myc, Oct3/4, Sox2, Nanog, and Lin28 are utilized. In other embodiments, Oct3/4, Sox2, c-Myc and Klf4 are utilized or Oct3/4, Sox2, Nanog, and Lin28.

Mouse and human cDNA sequences of these nuclear reprogramming substances are available with reference to the NCBI accession numbers mentioned in WO 2007/069666 and U.S. Pat. No. 8,183,038, which are incorporated herein by reference. Methods for introducing one or more reprogramming substances, or nucleic acids encoding these reprogramming substances, are known in the art, and disclosed for example, in U.S. Pat. Nos. 8,268,620, 8,691,574, 8,741,648, 8,546,140, in published U.S. Pat. Nos. 8,900,871 and 8,071,369, which are both incorporated herein by reference.

Once derived, iPSCs can be cultured in a medium sufficient to maintain pluripotency. The iPSCs may be used with various media and techniques developed to culture pluripotent stem cells, more specifically, embryonic stem cells, as described in U.S. Pat. No. 7,442,548 and U.S. Patent Pub. No. 2003/0211603. In the case of mouse cells, the culture is carried out with the addition of Leukemia Inhibitory Factor (LIF) as a differentiation suppression factor to an ordinary medium. In the case of human cells, it is desirable that basic fibroblast growth factor (bFGF) be added in place of LIF. Other methods for the culture and maintenance of iPSCs, as would be known to one of skill in the art, may be used with the methods disclosed herein.

In certain embodiments, undefined conditions may be used; for example, pluripotent cells may be cultured on fibroblast feeder cells or a medium that has been exposed to fibroblast feeder cells in order to maintain the stem cells in an undifferentiated state. In some embodiments, the cell is cultured in the co-presence of mouse embryonic fibroblasts treated with radiation or an antibiotic to terminate the cell division, as feeder cells. Alternately, pluripotent cells may be cultured and maintained in an essentially undifferentiated state using a defined, feeder-independent culture system, such as a TESR™ medium or E8™/Essential 8™ medium.

Plasmids have been designed with a number of goals in mind, such as achieving regulated high copy number and avoiding potential causes of plasmid instability in bacteria, and providing means for plasmid selection that are compatible with use in mammalian cells, including human cells. Particular attention has been paid to the dual requirements of plasmids for use in human cells. First, they are suitable for maintenance and fermentation in E. coli, so that large amounts of DNA can be produced and purified. Second, they are safe and suitable for use in human patients and animals. The first requirement calls for high copy number plasmids that can be selected for and stably maintained relatively easily during bacterial fermentation. The second requirement calls for attention to elements such as selectable markers and other coding sequences. In some embodiments, plasmids that encode a marker are composed of: (1) a high copy number replication origin, (2) a selectable marker, such as, but not limited to, the neo gene for antibiotic selection with kanamycin, (3) transcription termination sequences, including the tyrosinase enhancer and (4) a multicloning site for incorporation of various nucleic acid cassettes; and (5) a nucleic acid sequence encoding a marker operably linked to the tyrosinase promoter. In particular aspects, the plasmids do not comprise a tyrosinase enhancer or promoter. There are numerous plasmid vectors that are known in the art for inducing a nucleic acid encoding a protein. These include, but are not limited to, the vectors disclosed in U.S. Pat. Nos. 6,103,470; 7,598,364; 7,989,425; and 6,416,998, and U.S. application Ser. No. 12/478,154 which are incorporated herein by reference.

An episomal gene delivery system can be a plasmid, an Epstein-Barr virus (EBV)-based episomal vector, a yeast-based vector, an adenovirus-based vector, a simian virus 40 (SV40)-based episomal vector, a bovine papilloma virus (BPV)-based vector, or a lentiviral vector. A viral gene delivery system can be an RNA-based or DNA-based viral vector.

### IV. Engineering of Cells

In some embodiments, immune cells derived from the selected cord blood unit(s) are engineered by the hand of man to be utilized for a variety of purposes. The engineering may be for the purpose of clinical or research applications. The engineered immune cells may be stored, or they may be used, such as administered to an individual in need thereof, in some cases. The engineering may or may not be performed by the same individual that generated the immune cells from the selected cord blood unit(s).

In specific embodiments, the immune cells are engineered to express one or more non-natural receptors, such as antigen receptors. The antigen may be of any kind, and the engineering of the immune cell to express the antigen facilitates use of the cell for a clinical application, in at least some cases. The antigen may be a cancer antigen (including a tumor antigen or hematopoietic cell antigen), or the antigen may be with respect to a pathogen of any kind, including bacterial, viral, fungal, parasitic, and so forth.

The immune cells from the selected cord blood unit(s) (e.g., autologous or allogeneic T cells (e.g., regulatory T cells, CD4⁺ T cells, CD8⁺ T cells, or gamma-delta T cells), NK cells, invariant NK cells, NKT cells, stem cells (e.g., MSCs or iPS cells) can be genetically engineered to express antigen receptors such as engineered TCRs and/or CARs. For example, the immune cells may be modified to express a TCR having antigenic specificity for a cancer antigen. In particular embodiments, NK cells are engineered to express a TCR. The NK cells may be alternatively or further engineered to express a CAR. Multiple CARs and/or TCRs, such as to different antigens, may be added to a single cell type, such as T cells or NK cells.

Suitable methods of modification of cells or recombination reagents are known in the art. See, for instance, Sambrook and Ausubel, supra. For example, the cells may be transduced to express a TCR having antigenic specificity for a cancer antigen using transduction techniques described in Heemskerk et al., 2008 and Johnson et al., 2009.

In some embodiments, the cells comprise one or more nucleic acids introduced via genetic engineering that encode one or more antigen receptors, and genetically engineered products of such nucleic acids. In some embodiments, the nucleic acids are heterologous, i.e., normally not present in a cell or sample obtained from the cell, such as one obtained from another organism or cell, which for example, is not ordinarily found in the cell being engineered and/or an organism from which such cell is derived. In some embodiments, the nucleic acids are not naturally occurring, such as a nucleic acid not found in nature (e.g., chimeric).

In some embodiments, the CAR contains an extracellular antigen-recognition domain that specifically binds to an antigen. In some embodiments, the antigen is a protein expressed on the surface of cells. In some embodiments, the CAR is a TCR-like CAR and the antigen is a processed peptide antigen, such as a peptide antigen of an intracellular protein, which, like a TCR, is recognized on the cell surface in the context of a major histocompatibility complex (MHC) molecule.

Exemplary antigen receptors, including CARs and recombinant TCRs, as well as methods for engineering and introducing the receptors into cells, include those described, for example, in international patent application publication numbers WO2000/14257, WO2013/126726, WO2012/129514, WO2014/031687, WO2013/166321, WO2013/071154, WO2013/123061 U.S. patent application publication numbers US2002131960, US2013287748, US20130149337, U.S. Pat. Nos. 6,451,995, 7,446,190, 8,252,592, 8,339,645, 8,398,282, 7,446,179, 6,410,319, 7,070,995, 7,265,209, 7,354,762, 7,446,191, 8,324,353, and 8,479,118, and European patent application number EP2537416, and/or those described by Sadelain et al., 2013; Davila et al., 2013; Turtle et al., 2012; Wu et al., 2012. In some aspects, the genetically engineered antigen receptors include a CAR as described in U.S. Pat. No. 7,446,190, and those described in International Patent Application Publication No.: WO/2014055668 A1.

For embodiments in which TCRs are utilized, electroporation of RNA coding for the full length TCR alpha and beta (or gamma and delta) chains can be used as alternative to overcome long-term problems with autoreactivity caused by pairing of retrovirally transduced and endogenous TCR chains. Even if such alternative pairing takes place in the transient transfection strategy, the possibly generated autoreactive T cells will lose this autoreactivity after some time, because the introduced TCR .alpha. and .beta. chain are only transiently expressed. When the introduced TCR alpha and beta chain expression is diminished, only normal autologous T cells are left. This is not the case when full length TCR chains are introduced by stable retroviral transduction, which will never lose the introduced TCR chains, causing a constantly present autoreactivity in the patient.

Following genetic modification the immune cells may be immediately delivered (such as infused) or may be stored. In certain aspects, following genetic modification, the cells may be propagated for days, weeks, or months ex vivo as a bulk population within about 1, 2, 3, 4, 5 days or more following gene transfer into cells. In a further aspect, the transfectants are cloned and a clone demonstrating presence of a single integrated or episomally maintained expression cassette or plasmid, and expression of the chimeric receptor (as an example) is expanded ex vivo. The clone selected for expansion demonstrates the capacity to specifically recognize and lyse antigen-expressing target cells. The recombinant immune cells may be expanded by stimulation, such as with IL-2, or other cytokines that bind the common gamma-chain (e.g., IL-7, IL-12, IL-15, IL-21, and others). The recombinant immune cells may be expanded by stimulation with artificial antigen presenting cells. In a further aspect, the genetically modified cells may be cryopreserved.

A. Chimeric Antigen Receptors

In some embodiments, the immune cells are engineered to express a CAR, and the CAR may comprise: a) one or more intracellular signaling domains, b) a transmembrane domain, and c) an extracellular domain comprising an antigen binding region.

In some embodiments, the engineered antigen receptors include CARs, including activating or stimulatory CARs, costimulatory CARs (see WO2014/055668), and/or inhibitory CARs (iCARs, see Fedorov et al., 2013). The CARs generally include an extracellular antigen (or ligand) binding domain linked to one or more intracellular signaling components, in some aspects via linkers and/or transmembrane domain(s). Such molecules typically mimic or approximate a signal through a natural antigen receptor, a signal through such a receptor in combination with a costimulatory receptor, and/or a signal through a costimulatory receptor alone.

Certain embodiments of the present disclosure concern the use of nucleic acids, including nucleic acids encoding an antigen-specific CAR polypeptide, including a CAR that has been humanized to reduce immunogenicity (hCAR), comprising an intracellular signaling domain, a transmembrane domain, and an extracellular domain comprising one or more signaling motifs. In certain embodiments, the CAR may recognize an epitope comprising the shared space between one or more antigens. In certain embodiments, the binding region can comprise complementary determining regions of a monoclonal antibody, variable regions of a monoclonal antibody, and/or antigen binding fragments thereof. In another embodiment, that specificity is derived from a peptide (e.g., cytokine) that binds to a receptor.

It is contemplated that the human CAR nucleic acids may be human genes used to enhance cellular immunotherapy for human patients. In a specific embodiment, the invention includes a full-length CAR cDNA or coding region. The antigen binding regions or domain can comprise a fragment of the V_{H} and V_{L} chains of a single-chain variable fragment (scFv) derived from a particular human monoclonal antibody, such as those described in U.S. Pat. No. 7,109,304, incorporated herein by reference. The fragment can also be any number of different antigen binding domains of a human antigen-specific antibody. In a more specific embodiment, the fragment is an antigen-specific scFv encoded by a sequence that is optimized for human codon usage for expression in human cells.

The arrangement could be multimeric, such as a diabody or multimers. The multimers are most likely formed by cross pairing of the variable portion of the light and heavy chains into a diabody. The hinge portion of the construct can have multiple alternatives from being totally deleted, to having the first cysteine maintained, to a proline rather than a serine substitution, to being truncated up to the first cysteine. The Fc portion can be deleted. Any protein that is stable and/or dimerizes can serve this purpose. One could use just one of the Fc domains, e.g., either the CH2 or CH3 domain from human immunoglobulin. One could also use the hinge, CH2 and CH3 region of a human immunoglobulin that has been modified to improve dimerization. One could also use just the hinge portion of an immunoglobulin. One could also use portions of CD8alpha.

In some embodiments, the CAR nucleic acid comprises a sequence encoding other costimulatory receptors, such as a transmembrane domain and a modified CD28 intracellular signaling domain. Other costimulatory receptors include, but are not limited to one or more of CD28, CD27, OX-40 (CD134), DAP10, DAP12, and 4-1BB (CD137). In addition to a primary signal initiated by CD3zeta, an additional signal provided by a human costimulatory receptor inserted in a human CAR is important for full activation of NK cells and could help improve in vivo persistence and the therapeutic success of the adoptive immunotherapy.

In some embodiments, CAR is constructed with a specificity for a particular antigen (or marker or ligand), such as an antigen expressed in a particular cell type to be targeted by adoptive therapy, e.g., a cancer marker, and/or an antigen intended to induce a dampening response, such as an antigen expressed on a normal or non-diseased cell type. Thus, the CAR typically includes in its extracellular portion one or more antigen binding molecules, such as one or more antigen-binding fragment, domain, or portion, or one or more antibody variable domains, and/or antibody molecules. In some embodiments, the CAR includes an antigen-binding portion or portions of an antibody molecule, such as a single-chain antibody fragment (scFv) derived from the variable heavy (VH) and variable light (VL) chains of a monoclonal antibody (mAb).

In certain embodiments of the chimeric antigen receptor, the antigen-specific portion of the receptor (which may be referred to as an extracellular domain comprising an antigen binding region) comprises a tumor associated antigen or a pathogen-specific antigen binding domain. Antigens include carbohydrate antigens recognized by pattern-recognition receptors, such as Dectin-1. A tumor associated antigen may be of any kind so long as it is expressed on the cell surface of tumor cells. Exemplary embodiments of tumor associated antigens include CD19, CD20, carcinoembryonic antigen, alphafetoprotein, CA-125, MUC-1, CD56, EGFR, c-Met, AKT, Her2, Her3, epithelial tumor antigen, melanoma-associated antigen, mutated p53, mutated ras, and so forth. In certain embodiments, the CAR may be co-expressed with a cytokine to improve persistence when there is a low amount of tumor-associated antigen. For example, CAR may be co-expressed with IL-15.

The sequence of the open reading frame encoding the chimeric receptor can be obtained from a genomic DNA source, a cDNA source, or can be synthesized (e.g., via PCR), or combinations thereof. Depending upon the size of the genomic DNA and the number of introns, it may be desirable to use cDNA or a combination thereof as it is found that introns stabilize the mRNA. Also, it may be further advantageous to use endogenous or exogenous non-coding regions to stabilize the mRNA.

It is contemplated that the chimeric construct can be introduced into immune cells as naked DNA or in a suitable vector. Methods of stably transfecting cells by electroporation using naked DNA are known in the art. See, e.g., U.S. Pat. No. 6,410,319. Naked DNA generally refers to the DNA encoding a chimeric receptor contained in a plasmid expression vector in proper orientation for expression.

Alternatively, a viral vector (e.g., a retroviral vector, adenoviral vector, adeno-associated viral vector, or lentiviral vector) can be used to introduce the chimeric construct into immune cells. Suitable vectors for use in accordance with the method of the present disclosure are non-replicating in the immune cells. A large number of vectors are known that are based on viruses, where the copy number of the virus maintained in the cell is low enough to maintain the viability of the cell, such as, for example, vectors based on HIV, SV40, EBV, HSV, or BPV.

In some aspects, the antigen-specific binding, or recognition component is linked to one or more transmembrane and intracellular signaling domains. In some embodiments, the CAR includes a transmembrane domain fused to the extracellular domain of the CAR. In one embodiment, the transmembrane domain that naturally is associated with one of the domains in the CAR is used. In some instances, the transmembrane domain is selected or modified by amino acid substitution to avoid binding of such domains to the transmembrane domains of the same or different surface membrane proteins to minimize interactions with other members of the receptor complex.

The transmembrane domain in some embodiments is derived either from a natural or from a synthetic source. Where the source is natural, the domain in some aspects is derived from any membrane-bound or transmembrane protein. Transmembrane regions include those derived from (i.e. comprise at least the transmembrane region(s) of) the alpha, beta or zeta chain of the T-cell receptor, CD28, CD3 zeta, CD3 epsilon, CD3 gamma, CD3 delta, CD45, CD4, CD5, CD8, CD9, CD16, CD22, CD33, CD37, CD64, CD80, CD86, CD 134, CD137, CD154, ICOS/CD278, GITR/CD357, NKG2D, and DAP molecules. Alternatively the transmembrane domain in some embodiments is synthetic. In some aspects, the synthetic transmembrane domain comprises predominantly hydrophobic residues such as leucine and valine. In some aspects, a triplet of phenylalanine, tryptophan and valine will be found at each end of a synthetic transmembrane domain.

In certain embodiments, the platform technologies disclosed herein to genetically modify immune cells, such as NK cells, comprise (i) non-viral gene transfer using an electroporation device (e.g., a nucleofector), (ii) CARs that signal through endodomains (e.g., CD28/CD3-.zeta., CD137/CD3-zeta, or other combinations), (iii) CARs with variable lengths of extracellular domains connecting the antigen-recognition domain to the cell surface, and, in some cases, (iv) artificial antigen presenting cells (aAPC) derived from K562 to be able to robustly and numerically expand CAR.sup.+ immune cells (Singh et al., 2008; Singh et al., 2011).

B. T Cell Receptor (TCR)

In some embodiments, the genetically engineered antigen receptors include recombinant TCRs and/or TCRs cloned from naturally occurring T cells. A “T cell receptor” or “TCR” refers to a molecule that contains a variable .alpha. and .beta. chains (also known as TCR.alpha. and TCR.beta., respectively) or a variable .gamma. and .delta. chains (also known as TCR.gamma. and TCR.delta., respectively) and that is capable of specifically binding to an antigen peptide bound to a MHC receptor. In some embodiments, the TCR is in the .alpha..beta. form.

Typically, TCRs that exist in .alpha..beta. and .gamma..delta. forms are generally structurally similar, but T cells expressing them may have distinct anatomical locations or functions. A TCR can be found on the surface of a cell or in soluble form. Generally, a TCR is found on the surface of T cells (or T lymphocytes) where it is generally responsible for recognizing antigens bound to major histocompatibility complex (MHC) molecules. In some embodiments, a TCR also can contain a constant domain, a transmembrane domain and/or a short cytoplasmic tail (see, e.g., Janeway et al, 1997). For example, in some aspects, each chain of the TCR can possess one N-terminal immunoglobulin variable domain, one immunoglobulin constant domain, a transmembrane region, and a short cytoplasmic tail at the C-terminal end. In some embodiments, a TCR is associated with invariant proteins of the CD3 complex involved in mediating signal transduction. Unless otherwise stated, the term “TCR” should be understood to encompass functional TCR fragments thereof. The term also encompasses intact or full-length TCRs, including TCRs in the .alpha..beta. form or .gamma..delta. form.

Thus, for purposes herein, reference to a TCR includes any TCR or functional fragment, such as an antigen-binding portion of a TCR that binds to a specific antigenic peptide bound in an MHC molecule, i.e. MHC-peptide complex. An “antigen-binding portion” or antigen-binding fragment” of a TCR, which can be used interchangeably, refers to a molecule that contains a portion of the structural domains of a TCR, but that binds the antigen (e.g. MHC-peptide complex) to which the full TCR binds. In some cases, an antigen-binding portion contains the variable domains of a TCR, such as variable .alpha. chain and variable .beta. chain of a TCR, sufficient to form a binding site for binding to a specific MHC-peptide complex, such as generally where each chain contains three complementarity determining regions.

In some embodiments, the variable domains of the TCR chains associate to form loops, or complementarity determining regions (CDRs) analogous to immunoglobulins, which confer antigen recognition and determine peptide specificity by forming the binding site of the TCR molecule and determine peptide specificity. Typically, like immunoglobulins, the CDRs are separated by framework regions (FRs) (see, e.g., Jores et al., 1990; Chothia et al., 1988; Lefranc et al., 2003). In some embodiments, CDR3 is the main CDR responsible for recognizing processed antigen, although CDR1 of the alpha chain has also been shown to interact with the N-terminal part of the antigenic peptide, whereas CDR1 of the beta chain interacts with the C-terminal part of the peptide. CDR2 is thought to recognize the MHC molecule. In some embodiments, the variable region of the .beta.-chain can contain a further hypervariability (HV4) region.

In some embodiments, the TCR chains contain a constant domain. For example, like immunoglobulins, the extracellular portion of TCR chains (e.g., .alpha.-chain, .beta.-chain) can contain two immunoglobulin domains, a variable domain (e.g., Va or Vp; typically amino acids 1 to 116 based on Kabat numbering Kabat et al., “Sequences of Proteins of Immunological Interest, US Dept. Health and Human Services, Public Health Service National Institutes of Health, 1991, 5.sup.th ed.) at the N-terminus, and one constant domain (e.g., a-chain constant domain or C.sub.a, typically amino acids 117 to 259 based on Kabat, beta-chain constant domain or Cp, typically amino acids 117 to 295 based on Kabat) adjacent to the cell membrane. For example, in some cases, the extracellular portion of the TCR formed by the two chains contains two membrane-proximal constant domains, and two membrane-distal variable domains containing CDRs. The constant domain of the TCR domain contains short connecting sequences in which a cysteine residue forms a disulfide bond, making a link between the two chains. In some embodiments, a TCR may have an additional cysteine residue in each of the alpha and beta chains such that the TCR contains two disulfide bonds in the constant domains.

In some embodiments, the TCR chains can contain a transmembrane domain. In some embodiments, the transmembrane domain is positively charged. In some cases, the TCR chains contains a cytoplasmic tail. In some cases, the structure allows the TCR to associate with other molecules like CD3. For example, a TCR containing constant domains with a transmembrane region can anchor the protein in the cell membrane and associate with invariant subunits of the CD3 signaling apparatus or complex.

Generally, CD3 is a multi-protein complex that can possess three distinct chains (.gamma., .delta., and .epsilon.) in mammals and the .zeta.-chain. For example, in mammals the complex can contain a CD3-gamma chain, a CD3-delta chain, two CD3-epsilon chains, and a homodimer of CD3-zeta chains. The CD3-gamma, CD3-delta, and CD3-epsilon chains are highly related cell surface proteins of the immunoglobulin superfamily containing a single immunoglobulin domain. The transmembrane regions of the CD3-gamma, CD3-delta, and CD3-epsilon chains are negatively charged, which is a characteristic that allows these chains to associate with the positively charged T cell receptor chains. The intracellular tails of the CD3-gamma, CD3-delta, and CD3-epsilon chains each contain a single conserved motif known as an immunoreceptor tyrosine-based activation motif or ITAM, whereas each CD3-zeta chain has three. Generally, ITAMs are involved in the signaling capacity of the TCR complex. These accessory molecules have negatively charged transmembrane regions and play a role in propagating the signal from the TCR into the cell.

In some embodiments, the TCR may be a heterodimer of two chains alpha and beta (or optionally gamma and delta) or it may be a single chain TCR construct. In some embodiments, the TCR is a heterodimer containing two separate chains (alpha and beta chains or gamma and delta chains) that are linked, such as by a disulfide bond or disulfide bonds. In some embodiments, a TCR for a target antigen (e.g., a cancer antigen) is identified and introduced into the cells. In some embodiments, nucleic acid encoding the TCR can be obtained from a variety of sources, such as by polymerase chain reaction (PCR) amplification of publicly available TCR DNA sequences. In some embodiments, the TCR is obtained from a biological source, such as from cells such as from a T cell (e.g. cytotoxic T cell), T cell hybridomas or other publicly available source. In some embodiments, the T cells can be obtained from in vivo isolated cells. In some embodiments, a high-affinity T cell clone can be isolated from a patient, and the TCR isolated. In some embodiments, the T cells can be a cultured T cell hybridoma or clone. In some embodiments, the TCR clone for a target antigen has been generated in transgenic mice engineered with human immune system genes (e.g., the human leukocyte antigen system, or HLA). See, e.g., tumor antigens (see, e.g., Parkhurst et al., 2009 and Cohen et al., 2005). In some embodiments, phage display is used to isolate TCRs against a target antigen (see, e.g., Varela-Rohena et al., 2008 and Li, 2005). In some embodiments, the TCR or antigen-binding portion thereof can be synthetically generated from knowledge of the sequence of the TCR.

C. Antigens

In specific cases, the immune cells derived from the selected cord blood unit(s) are engineered to express a protein that targets an antigen, such as a receptor that targets an antigen. In specific cases, the receptor is genetically engineered to comprise chimeric components from different sources. Among the antigens targeted by the genetically engineered antigen receptors are those expressed in the context of a disease, condition, or cell type to be targeted via the adoptive cell therapy. Among the diseases and conditions are proliferative, neoplastic, and malignant diseases and disorders, including cancers and tumors, including hematologic cancers, cancers of the immune system, such as lymphomas, leukemias, and/or myelomas, such as B, T, and myeloid leukemias, lymphomas, and multiple myelomas. In some embodiments, the antigen is selectively expressed or overexpressed on cells of the disease or condition, e.g., the tumor or pathogenic cells, as compared to normal or non-targeted cells or tissues. In other embodiments, the antigen is expressed on normal cells and/or is expressed on the engineered cells.

Any suitable antigen may find use in the present method. Exemplary antigens include, but are not limited to, antigenic molecules from infectious agents, auto-/self-antigens, tumor-/cancer-associated antigens, and tumor neoantigens (Linnemann et al., 2015). In particular aspects, the antigens include NY-ESO, EGFRvIII, Muc-1, Her2, CA-125, WT-1, Mage-A3, Mage-A4, Mage-A10, TRAIL/DR4, and CEA. In particular aspects, the antigens for the two or more antigen receptors include, but are not limited to, CD19, EBNA, WT1, CD123, NY-ESO, EGFRvIII, MUC1, HER2, CA-125, WT1, Mage-A3, Mage-A4, Mage-A10, TRAIL/DR4, and/or CEA. The sequences for these antigens are known in the art, for example, CD19 (Accession No. NG_007275.1), EBNA (Accession No. NG_002392.2), WT1 (Accession No. NG_009272.1), CD123 (Accession No. NC_000023.11), NY-ESO (Accession No. NC_000023.11), EGFRvIII (Accession No. NG_007726.3), MUC1 (Accession No. NG_029383.1), HER2 (Accession No. NG_007503.1), CA-125 (Accession No. NG_055257.1), WT1 (Accession No. NG_009272.1), Mage-A3 (Accession No. NG_013244.1), Mage-A4 (Accession No. NG_013245.1), Mage-A10 (Accession No. NC_000023.11), TRAIL/DR4 (Accession No. NC_000003.12), and/or CEA (Accession No. NC_000019.10).

Tumor-associated antigens may be derived from prostate, breast, colorectal, lung, pancreatic, renal, mesothelioma, ovarian, or melanoma cancers. Exemplary tumor-associated antigens or tumor cell-derived antigens include MAGE 1, 3, and MAGE 4 (or other MAGE antigens such as those disclosed in International Patent Publication No. WO99/40188); PRAME; BAGE; RAGE, Lage (also known as NY ESO 1); SAGE; and HAGE or GAGE. These non-limiting examples of tumor antigens are expressed in a wide range of tumor types such as melanoma, lung carcinoma, sarcoma, and bladder carcinoma. See, e.g., U.S. Pat. No. 6,544,518. Prostate cancer tumor-associated antigens include, for example, prostate specific membrane antigen (PSMA), prostate-specific antigen (PSA), prostatic acid phosphates, NKX3.1, and six-transmembrane epithelial antigen of the prostate (STEAP).

Other tumor associated antigens include Plu-1, HASH-1, HasH-2, Cripto and Criptin. Additionally, a tumor antigen may be a self peptide hormone, such as whole length gonadotrophin hormone releasing hormone (GnRH), a short 10 amino acid long peptide, useful in the treatment of many cancers.

Tumor antigens include tumor antigens derived from cancers that are characterized by tumor-associated antigen expression, such as HER-2/neu expression. Tumor-associated antigens of interest include lineage-specific tumor antigens such as the melanocyte-melanoma lineage antigens MART-1/Melan-A, gp100, gp75, mda-7, tyrosinase and tyrosinase-related protein. Illustrative tumor-associated antigens include, but are not limited to, tumor antigens derived from or comprising any one or more of, p53, Ras, c-Myc, cytoplasmic serine/threonine kinases (e.g., A-Raf, B-Raf, and C-Raf, cyclin-dependent kinases), MAGE-A1, MAGE-A2, MAGE-A3, MAGE-A4, MAGE-A6, MAGE-A10, MAGE-A12, MART-1, BAGE, DAM-6, -10, GAGE-1, -2, -8, GAGE-3, -4, -5, -6, -7B, NA88-A, MART-1, MC1R, Gp100, PSA, PSM, Tyrosinase, TRP-1, TRP-2, ART-4, CAMEL, CEA, Cyp-B, hTERT, hTRT, iCE, MUC1, MUC2, Phosphoinositide 3-kinases (PI3Ks), TRK receptors, PRAME, P15, RU1, RU2, SART-1, SART-3, Wilms' tumor antigen (WT1), AFP, -catenin/m, Caspase-8/m, CEA, CDK-4/m, ELF2M, GnT-V, G250, HSP70-2M, HST-2, KIAA0205, MUM-1, MUM-2, MUM-3, Myosin/m, RAGE, SART-2, TRP-2/INT2, 707-AP, Annexin II, CDC27/m, TPI/mbcr-abl, BCR-ABL, interferon regulatory factor 4 (IRF4), ETV6/AML, LDLR/FUT, Pml/RAR, Tumor-associated calcium signal transducer 1 (TACSTD1) TACSTD2, receptor tyrosine kinases (e.g., Epidermal Growth Factor receptor (EGFR) (in particular, EGFRvIII), platelet derived growth factor receptor (PDGFR), vascular endothelial growth factor receptor (VEGFR)), cytoplasmic tyrosine kinases (e.g., src-family, syk-ZAP70 family), integrin-linked kinase (ILK), signal transducers and activators of transcription STAT3, STATS, and STATE, hypoxia inducible factors (e.g., HIF-1 and HIF-2), Nuclear Factor-Kappa B (NF-B), Notch receptors (e.g., Notchl-4), c-Met, mammalian targets of rapamycin (mTOR), WNT, extracellular signal-regulated kinases (ERKs), and their regulatory subunits, PMSA, PR-3, MDM2, Mesothelin, renal cell carcinoma-5T4, SM22-alpha, carbonic anhydrases I (CAI) and IX (CAIX) (also known as G250), STEAD, TEL/AML1, GD2, proteinase3, hTERT, sarcoma translocation breakpoints, EphA2, ML-IAP, EpCAM, ERG (TMPRSS2 ETS fusion gene), NA17, PAX3, ALK, androgen receptor, cyclin B1, polysialic acid, MYCN, RhoC, GD3, fucosyl GM1, mesothelian, PSCA, sLe, PLAC1, GM3, BORIS, Tn, GLoboH, NY-BR-1, RGsS, SART3, STn, PAX5, OY-TES1, sperm protein 17, LCK, HMWMAA, AKAP-4, SSX2, XAGE 1, B7H3, legumain, TIE2, Page4, MAD-CT-1, FAP, MAD-CT-2, fos related antigen 1, CBX2, CLDN6, SPANX, TPTE, ACTL8, ANKRD30A, CDKN2A, MAD2L1, CTAGiB, SUNC1, LRRN1 and idiotype.

Antigens may include epitopic regions or epitopic peptides derived from genes mutated in tumor cells or from genes transcribed at different levels in tumor cells compared to normal cells, such as telomerase enzyme, survivin, mesothelin, mutated ras, bcr/abl rearrangement, Her2/neu, mutated or wild-type p53, cytochrome P450 1B1, and abnormally expressed intron sequences such as N-acetylglucosaminyltransferase-V; clonal rearrangements of immunoglobulin genes generating unique idiotypes in myeloma and B-cell lymphomas; tumor antigens that include epitopic regions or epitopic peptides derived from oncoviral processes, such as human papilloma virus proteins E6 and E7; Epstein bar virus protein LMP2; nonmutated oncofetal proteins with a tumor-selective expression, such as carcinoembryonic antigen and alpha-fetoprotein.

In other embodiments, an antigen is obtained or derived from a pathogenic microorganism or from an opportunistic pathogenic microorganism (also called herein an infectious disease microorganism), such as a virus, fungus, parasite, and bacterium. In certain embodiments, antigens derived from such a microorganism include full-length proteins.

Illustrative pathogenic organisms whose antigens are contemplated for use in the method described herein include coronavirus of any kind, including SARS-CoV and SARS-CoV2, human immunodeficiency virus (HIV), herpes simplex virus (HSV), respiratory syncytial virus (RSV), cytomegalovirus (CMV), Epstein-Barr virus (EBV), Influenza A, B, and C, vesicular stomatitis virus (VSV), vesicular stomatitis virus (VSV), polyomavirus (e.g., BK virus and JC virus), adenovirus, Staphylococcus species including Methicillin-resistant Staphylococcus aureus (MRSA), and Streptococcus species, including Streptococcus pneumoniae. As would be understood by the skilled person, proteins derived from these and other pathogenic microorganisms for use as antigen as described herein and nucleotide sequences encoding the proteins may be identified in publications and in public databases such as GENBANK®, SWISS-PROT®, and TREMBL®.

Antigens derived from human immunodeficiency virus (HIV) include any of the HIV virion structural proteins (e.g., gp120, gp41, p17, p24), protease, reverse transcriptase, or HIV proteins encoded by tat, rev, nef, vif, vpr and vpu.

Antigens derived from herpes simplex virus (e.g., HSV 1 and HSV2) include, but are not limited to, proteins expressed from HSV late genes. The late group of genes predominantly encodes proteins that form the virion particle. Such proteins include the five proteins from (UL) which form the viral capsid: UL6, UL18, UL35, UL38 and the major capsid protein UL19, UL45, and UL27, each of which may be used as an antigen as described herein. Other illustrative HSV proteins contemplated for use as antigens herein include the ICP27 (H1, H2), glycoprotein B (gB) and glycoprotein D (gD) proteins. The HSV genome comprises at least 74 genes, each encoding a protein that could potentially be used as an antigen.

Antigens derived from cytomegalovirus (CMV) include CMV structural proteins, viral antigens expressed during the immediate early and early phases of virus replication, glycoproteins I and III, capsid protein, coat protein, lower matrix protein pp65 (ppUL83), p52 (ppUL44), IE1 and IE2 (UL123 and UL122), protein products from the cluster of genes from UL128-UL150 (Rykman, et al., 2006), envelope glycoprotein B (gB), gH, gN, and pp150. As would be understood by the skilled person, CMV proteins for use as antigens described herein may be identified in public databases such as GENBANK®, SWISS-PROT®, and TREMBL® (see e.g., Bennekov et al., 2004; Loewendorf et al., 2010; Marschall et al., 2009).

Antigens derived from Epstein-Ban virus (EBV) that are contemplated for use in certain embodiments include EBV lytic proteins gp350 and gp110, EBV proteins produced during latent cycle infection including Epstein-Ban nuclear antigen (EBNA)-1, EBNA-2, EBNA-3A, EBNA-3B, EBNA-3C, EBNA-leader protein (EBNA-LP) and latent membrane proteins (LMP)-1, LMP-2A and LMP-2B (see, e.g., Lockey et al., 2008).

Antigens derived from respiratory syncytial virus (RSV) that are contemplated for use herein include any of the eleven proteins encoded by the RSV genome, or antigenic fragments thereof: NS 1, NS2, N (nucleocapsid protein), M (Matrix protein) SH, G and F (viral coat proteins), M2 (second matrix protein), M2-1 (elongation factor), M2-2 (transcription regulation), RNA polymerase, and phosphoprotein P.

Antigens derived from Vesicular stomatitis virus (VSV) that are contemplated for use include any one of the five major proteins encoded by the VSV genome, and antigenic fragments thereof: large protein (L), glycoprotein (G), nucleoprotein (N), phosphoprotein (P), and matrix protein (M) (see, e.g., Rieder et al., 1999).

Antigens derived from an influenza virus that are contemplated for use in certain embodiments include hemagglutinin (HA), neuraminidase (NA), nucleoprotein (NP), matrix proteins M1 and M2, NS1, NS2 (NEP), PA, PB1, PB1-F2, and PB2.

Exemplary viral antigens also include, but are not limited to, adenovirus polypeptides, alphavirus polypeptides, calicivirus polypeptides (e.g., a calicivirus capsid antigen), coronavirus polypeptides, distemper virus polypeptides, Ebola virus polypeptides, enterovirus polypeptides, flavivirus polypeptides, hepatitis virus (AE) polypeptides (a hepatitis B core or surface antigen, a hepatitis C virus E1 or E2 glycoproteins, core, or non-structural proteins), herpesvirus polypeptides (including a herpes simplex virus or varicella zoster virus glycoprotein), infectious peritonitis virus polypeptides, leukemia virus polypeptides, Marburg virus polypeptides, orthomyxovirus polypeptides, papilloma virus polypeptides, parainfluenza virus polypeptides (e.g., the hemagglutinin and neuraminidase polypeptides), paramyxovirus polypeptides, parvovirus polypeptides, pestivirus polypeptides, picorna virus polypeptides (e.g., a poliovirus capsid polypeptide), pox virus polypeptides (e.g., a vaccinia virus polypeptide), rabies virus polypeptides (e.g., a rabies virus glycoprotein G), reovirus polypeptides, retrovirus polypeptides, and rotavirus polypeptides.

In certain embodiments, the antigen may be bacterial antigens. In certain embodiments, a bacterial antigen of interest may be a secreted polypeptide. In other certain embodiments, bacterial antigens include antigens that have a portion or portions of the polypeptide exposed on the outer cell surface of the bacteria.

Antigens derived from Staphylococcus species including Methicillin-resistant Staphylococcus aureus (MRSA) that are contemplated for use include virulence regulators, such as the Agr system, Sar and Sae, the Arl system, Sar homologues (Rot, MgrA, SarS, SarR, SarT, SarU, SarV, SarX, SarZ and TcaR), the Srr system and TRAP. Other Staphylococcus proteins that may serve as antigens include Clp proteins, HtrA, MsrR, aconitase, CcpA, SvrA, Msa, CfvA and CfvB (see, e.g., Staphylococcus: Molecular Genetics, 2008 Caister Academic Press, Ed. Jodi Lindsay). The genomes for two species of Staphylococcus aureus (N315 and Mu50) have been sequenced and are publicly available, for example at PATRIC (PATRIC: The VBI PathoSystems Resource Integration Center, Snyder et al., 2007). As would be understood by the skilled person, Staphylococcus proteins for use as antigens may also be identified in other public databases such as GenBank®, Swiss-Prot®, and TrEMBL®.

Antigens derived from Streptococcus pneumoniae that are contemplated for use in certain embodiments described herein include pneumolysin, PspA, choline-binding protein A (CbpA), NanA, NanB, SpnHL, PavA, LytA, Pht, and pilin proteins (RrgA; RrgB; RrgC). Antigenic proteins of Streptococcus pneumoniae are also known in the art and may be used as an antigen in some embodiments (see, e.g., Zysk et al., 2000). The complete genome sequence of a virulent strain of Streptococcus pneumoniae has been sequenced and, as would be understood by the skilled person, S. pneumoniae proteins for use herein may also be identified in other public databases such as GENBANK®, SWISS-PROT®, and TREMBL®. Proteins of particular interest for antigens according to the present disclosure include virulence factors and proteins predicted to be exposed at the surface of the pneumococci (see, e.g., Frolet et al., 2010).

Examples of bacterial antigens that may be used as antigens include, but are not limited to, Actinomyces polypeptides, Bacillus polypeptides, Bacteroides polypeptides, Bordetella polypeptides, Bartonella polypeptides, Borrelia polypeptides (e.g., B. burgdorferi OspA), Brucella polypeptides, Campylobacter polypeptides, Capnocytophaga polypeptides, Chlamydia polypeptides, Corynebacterium polypeptides, Coxiella polypeptides, Dermatophilus polypeptides, Enterococcus polypeptides, Ehrlichia polypeptides, Escherichia polypeptides, Francisella polypeptides, Fusobacterium polypeptides, Haemobartonella polypeptides, Haemophilus polypeptides (e.g., H. influenzae type b outer membrane protein), Helicobacter polypeptides, Klebsiella polypeptides, L-form bacteria polypeptides, Leptospira polypeptides, Listeria polypeptides, Mycobacteria polypeptides, Mycoplasma polypeptides, Neisseria polypeptides, Neorickettsia polypeptides, Nocardia polypeptides, Pasteurella polypeptides, Peptococcus polypeptides, Peptostreptococcus polypeptides, Pneumococcus polypeptides (i.e., S. pneumoniae polypeptides) (see description herein), Proteus polypeptides, Pseudomonas polypeptides, Rickettsia polypeptides, Rochalimaea polypeptides, Salmonella polypeptides, Shigella polypeptides, Staphylococcus polypeptides, group A streptococcus polypeptides (e.g., S. pyogenes M proteins), group B streptococcus (S. agalactiae) polypeptides, Treponema polypeptides, and Yersinia polypeptides (e.g., Y. pestis F1 and V antigens).

Examples of fungal antigens include, but are not limited to, Absidia polypeptides, Acremonium polypeptides, Alternaria polypeptides, Aspergillus polypeptides, Basidiobolus polypeptides, Bipolaris polypeptides, Blastomyces polypeptides, Candida polypeptides, Coccidioides polypeptides, Conidiobolus polypeptides, Cryptococcus polypeptides, Curvalaria polypeptides, Epidermophyton polypeptides, Exophiala polypeptides, Geotrichum polypeptides, Histoplasma polypeptides, Madurella polypeptides, Malassezia polypeptides, Microsporum polypeptides, Moniliella polypeptides, Mortierella polypeptides, Mucor polypeptides, Paecilomyces polypeptides, Penicillium polypeptides, Phialemonium polypeptides, Phialophora polypeptides, Prototheca polypeptides, Pseudallescheria polypeptides, Pseudomicrodochium polypeptides, Pythium polypeptides, Rhinosporidium polypeptides, Rhizopus polypeptides, Scolecobasidium polypeptides, Sporothrix polypeptides, Stemphylium polypeptides, Trichophyton polypeptides, Trichosporon polypeptides, and Xylohypha polypeptides.

Examples of protozoan parasite antigens include, but are not limited to, Babesia polypeptides, Balantidium polypeptides, Besnoitia polypeptides, Cryptosporidium polypeptides, Eimeria polypeptides, Encephalitozoon polypeptides, Entamoeba polypeptides, Giardia polypeptides, Hammondia polypeptides, Hepatozoon polypeptides, Isospora polypeptides, Leishmania polypeptides, Microsporidia polypeptides, Neospora polypeptides, Nosema polypeptides, Pentatrichomonas polypeptides, Plasmodium polypeptides. Examples of helminth parasite antigens include, but are not limited to, Acanthocheilonema polypeptides, Aelurostrongylus polypeptides, Ancylostoma polypeptides, Angiostrongylus polypeptides, Ascaris polypeptides, Brugia polypeptides, Bunostomum polypeptides, Capillaria polypeptides, Chabertia polypeptides, Cooperia polypeptides, Crenosoma polypeptides, Dictyocaulus polypeptides, Dioctophyme polypeptides, Dipetalonema polypeptides, Diphyllobothrium polypeptides, Diplydium polypeptides, Dirofilaria polypeptides, Dracunculus polypeptides, Enterobius polypeptides, Filaroides polypeptides, Haemonchus polypeptides, Lagochilascaris polypeptides, Loa polypeptides, Mansonella polypeptides, Muellerius polypeptides, Nanophyetus polypeptides, Necator polypeptides, Nematodirus polypeptides, Oesophagostomum polypeptides, Onchocerca polypeptides, Opisthorchis polypeptides, Ostertagia polypeptides, Parafilaria polypeptides, Paragonimus polypeptides, Parascaris polypeptides, Physaloptera polypeptides, Protostrongylus polypeptides, Setaria polypeptides, Spirocerca polypeptides Spirometra polypeptides, Stephanofilaria polypeptides, Strongyloides polypeptides, Strongylus polypeptides, Thelazia polypeptides, Toxascaris polypeptides, Toxocara polypeptides, Trichinella polypeptides, Trichostrongylus polypeptides, Trichuris polypeptides, Uncinaria polypeptides, and Wuchereria polypeptides. (e.g., P. falciparum circumsporozoite (PfCSP)), sporozoite surface protein 2 (PfSSP2), carboxyl terminus of liver state antigen 1 (PfLSA1 c-term), and exported protein 1 (PfExp-1), Pneumocystis polypeptides, Sarcocystis polypeptides, Schistosoma polypeptides, Theileria polypeptides, Toxoplasma polypeptides, and Trypanosoma polypeptides.

Examples of ectoparasite antigens include, but are not limited to, polypeptides (including antigens as well as allergens) from fleas; ticks, including hard ticks and soft ticks; flies, such as midges, mosquitoes, sand flies, black flies, horse flies, horn flies, deer flies, tsetse flies, stable flies, myiasis-causing flies and biting gnats; ants; spiders, lice; mites; and true bugs, such as bed bugs and kissing bugs.

D. Cytokines

In some cases, immune cells derived from the selected cord blood unit(s) are engineered to express one or more cytokines, including one or more heterologous cytokines. The cytokines may be of any kind, but in specific embodiments, the heterologous cytokine(s) is selected from the group consisting of IL-4, IL-10, IL-7, IL-2, IL-15, IL-12, IL-18, IL-21, and a combination thereof.

In specific embodiments, the cytokine is IL-15. IL-15 is tissue-restricted and only under pathologic conditions is it observed at any level in the serum, or systemically. IL-15 possesses several attributes that are desirable for adoptive therapy. IL-15 is a homeostatic cytokine that induces development and cell proliferation of natural killer cells, promotes the eradication of established tumors via alleviating functional suppression of tumor-resident cells, and inhibits AICD.

In one embodiments, the present disclosure concerns co-modifying immune cells expressing CAR and/or TCR immune cells with one or more cytokines, including IL-15. In addition to IL-15, other cytokines are envisioned. These include, but are not limited to, cytokines, chemokines, and other molecules that contribute to the activation and proliferation of cells used for human application. NK or T cells expressing IL-15 are capable of continued supportive cytokine signaling, which is critical to their survival post-infusion.

E. Suicide Genes

The immune cells of the present disclosure derived from cord blood unit(s) may comprise one or more suicide genes. The term “suicide gene” as used herein is defined as a gene which, upon administration of a prodrug, effects transition of a gene product to a compound which kills its host cell. Examples of suicide gene/prodrug combinations which may be used are Herpes Simplex Virus-thymidine kinase (HSV-tk) and ganciclovir, acyclovir, or FIAU; oxidoreductase and cycloheximide; cytosine deaminase and 5-fluorocytosine; thymidine kinase thymidilate kinase (Tdk::Tmk) and AZT; and deoxycytidine kinase and cytosine arabinoside.

The E. coli purine nucleoside phosphorylase, a so-called suicide gene which converts the prodrug 6-methylpurine deoxyriboside to toxic purine 6-methylpurine. Other examples of suicide genes used with prodrug therapy are the E. coli cytosine deaminase gene and the HSV thymidine kinase gene.

Exemplary suicide genes include CD20, CD52, EGFRv3, or inducible caspase 9. In one embodiment, a truncated version of EGFR variant III (EGFRv3) may be used as a suicide antigen which can be ablated by Cetuximab. Further suicide genes known in the art that may be used in the present disclosure include Purine nucleoside phosphorylase (PNP), Cytochrome p450 enzymes (CYP), Carboxypeptidases (CP), Carboxylesterase (CE), Nitroreductase (NTR), Guanine Ribosyltransferase (XGRTP), Glycosidase enzymes, Methionine-.alpha.,.gamma.-lyase (MET), and Thymidine phosphorylase (TP).

F. Gene Disruption

In some embodiments, the immune cells are engineered to have disruption of expression of one or more endogenous genes. The disruption may be a knockout or knockdown, in specific cases. The disruption may be produced in the cells by any suitable method, including CRISPR, antisense technology, such as RNAi, siRNA, shRNA, and/or ribozymes, which generally result in transient reduction of expression, as well as gene editing techniques that result in targeted gene inactivation or disruption, e.g., by induction of breaks and/or homologous recombination.

In particular cases, one or more endogenous genes of the immune cells are modified, such as disrupted in expression where the expression is reduced in part or in full. In specific cases, one or more genes are knocked down or knocked out. In specific cases, multiple genes are knocked down or knocked out in the same step or in multiple steps. The genes that are edited in the immune cells may be of any kind. In specific cases the genes that are edited in the immune cells allow the immune cells to work more effectively in a tumor microenvironment. In specific cases, the genes are one or more of NKG2A, SIGLEC-7, LAG3, TIM3, CISH, FOXO1, TGFBR2, TIGIT, CD96, ADORA2, NR3C1, PD1, PDL-1, PDL-2, CD47, SIRPA, SHIP1, ADAM17, RPS6, 4EBP1, CD25, CD40, IL21R, ICAM1, CD95, CD80, CD86, IL10R, TDAG8, CD5, CD7, SLAMF7, CD38, LAG3, TCR, beta2-microglubulin, HLA, CD73, and CD39. In certain embodiments, an endogenous gene that is disrupted by CRISPR is TIGIT, and in specific cases a gRNA utilized for this is GACAGGCACAATAGAAACAA (SEQ ID NO:1). In some embodiments, an endogenous gene that is edited by CRISPR is CD38, and in specific cases a gRNA utilized for this is TGAGTTCCCAACTTCATTAG (SEQ ID NO:2) and/or GCGGGACATGTTCACCCTGG (SEQ ID NO:3).

### V. Methods of Use

Once the cord blood unit(s) are selected, immune cells derived therefrom may or may not be engineered and may or may not be stored. In any event, a therapeutically effective of the immune cells, engineered or not, may be delivered to an individual in need thereof. The immune cells are particularly effective because they have been derived from selected cord blood unit(s) for the explicit reason of having met one or more selection criteria, as described herein.

In some embodiments, the present disclosure provides methods for immunotherapy comprising administering an effective amount of the immune cells produced by methods the present disclosure. In one embodiments, a medical disease or disorder is treated by transfer of an immune cell population that elicits an immune response. In certain embodiments of the present disclosure, cancer or infection is treated by transfer of the produced immune cell population that elicits an immune response. Provided herein are methods for treating or delaying progression of cancer in an individual comprising administering to the individual an effective amount an antigen-specific cell therapy. The present methods may be applied for the treatment of immune disorders, solid cancers, hematologic cancers, and viral infections.

Tumors for which the present treatment methods are useful include any malignant cell type, such as those found in a solid tumor or a hematological tumor. Exemplary solid tumors can include, but are not limited to, a tumor of an organ selected from the group consisting of pancreas, colon, cecum, stomach, brain, head, neck, ovary, kidney, larynx, sarcoma, lung, bladder, melanoma, prostate, and breast. Exemplary hematological tumors include tumors of the bone marrow, T or B cell malignancies, leukemias, lymphomas, blastomas, myelomas, and the like. Further examples of cancers that may be treated using the methods provided herein include, but are not limited to, lung cancer (including small-cell lung cancer, non-small cell lung cancer, adenocarcinoma of the lung, and squamous carcinoma of the lung), cancer of the peritoneum, gastric or stomach cancer (including gastrointestinal cancer and gastrointestinal stromal cancer), pancreatic cancer, cervical cancer, ovarian cancer, liver cancer, bladder cancer, breast cancer, colon cancer, colorectal cancer, endometrial or uterine carcinoma, salivary gland carcinoma, kidney or renal cancer, prostate cancer, vulval cancer, thyroid cancer, various types of head and neck cancer, and melanoma.

The cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; lentigo malignant melanoma; acral lentiginous melanomas; nodular melanomas; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; hodgkin's disease; hodgkin's; paragranuloma; malignant lymphoma, small lymphocytic; malignant lymphoma, large cell, diffuse; malignant lymphoma, follicular; mycosis fungoides; other specified non-hodgkin's lymphomas; B-cell lymphoma; low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; Waldenstrom's macroglobulinemia; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; hairy cell leukemia; chronic lymphocytic leukemia (CLL); acute lymphoblastic leukemia (ALL); acute myeloid leukemia (AML); and chronic myeloblastic leukemia.

Particular embodiments concern methods of treatment of leukemia. Leukemia is a cancer of the blood or bone marrow and is characterized by an abnormal proliferation (production by multiplication) of blood cells, usually white blood cells (leukocytes). It is part of the broad group of diseases called hematological neoplasms. Leukemia is a broad term covering a spectrum of diseases. Leukemia is clinically and pathologically split into its acute and chronic forms.

In certain embodiments of the present disclosure, immune cells are delivered to an individual in need thereof, such as an individual that has cancer or an infection. The cells then enhance the individual's immune system to attack the respective cancer or pathogenic cells. In some cases, the individual is provided with one or more doses of the immune cells. In cases where the individual is provided with two or more doses of the immune cells, the duration between the administrations should be sufficient to allow time for propagation in the individual, and in specific embodiments the duration between doses is 1, 2, 3, 4, 5, 6, 7, or more days.

Certain embodiments of the present disclosure provide methods for treating or preventing an immune-mediated disorder. In one embodiment, the subject has an autoimmune disease. Non-limiting examples of autoimmune diseases include: alopecia areata, ankylosing spondylitis, antiphospholipid syndrome, autoimmune Addison's disease, autoimmune diseases of the adrenal gland, autoimmune hemolytic anemia, autoimmune hepatitis, autoimmune oophoritis and orchitis, autoimmune thrombocytopenia, Behcet's disease, bullous pemphigoid, cardiomyopathy, celiac spate-dermatitis, chronic fatigue immune dysfunction syndrome (CFIDS), chronic inflammatory demyelinating polyneuropathy, Churg-Strauss syndrome, cicatrical pemphigoid, CREST syndrome, cold agglutinin disease, Crohn's disease, discoid lupus, essential mixed cryoglobulinemia, fibromyalgia-fibromyositis, glomerulonephritis, Graves' disease, Guillain-Barre, Hashimoto's thyroiditis, idiopathic pulmonary fibrosis, idiopathic thrombocytopenia purpura (ITP), IgA neuropathy, juvenile arthritis, lichen planus, lupus erthematosus, Meniere's disease, mixed connective tissue disease, multiple sclerosis, type 1 or immune-mediated diabetes mellitus, myasthenia gravis, nephrotic syndrome (such as minimal change disease, focal glomerulosclerosis, or membranous nephropathy), pemphigus vulgaris, pernicious anemia, polyarteritis nodosa, polychondritis, polyglandular syndromes, polymyalgia rheumatica, polymyositis and dermatomyositis, primary agammaglobulinemia, primary biliary cirrhosis, psoriasis, psoriatic arthritis, Raynaud's phenomenon, Reiter's syndrome, Rheumatoid arthritis, sarcoidosis, scleroderma, Sjogren's syndrome, stiff-man syndrome, systemic lupus erythematosus, lupus erythematosus, ulcerative colitis, uveitis, vasculitides (such as polyarteritis nodosa, takayasu arteritis, temporal arteritis/giant cell arteritis, or dermatitis herpetiformis vasculitis), vitiligo, and Wegener's granulomatosis. Thus, some examples of an autoimmune disease that can be treated using the methods disclosed herein include, but are not limited to, multiple sclerosis, rheumatoid arthritis, systemic lupus erythematosis, type I diabetes mellitus, Crohn's disease; ulcerative colitis, myasthenia gravis, glomerulonephritis, ankylosing spondylitis, vasculitis, or psoriasis. The subject can also have an allergic disorder such as Asthma.

In yet another embodiment, the subject is the recipient of a transplanted organ or stem cells and immune cells are used to prevent and/or treat rejection. In particular embodiments, the subject has or is at risk of developing graft versus host disease. GVHD is a possible complication of any transplant that uses or contains stem cells from either a related or an unrelated donor. There are two kinds of GVHD, acute and chronic. Acute GVHD appears within the first three months following transplantation. Signs of acute GVHD include a reddish skin rash on the hands and feet that may spread and become more severe, with peeling or blistering skin. Acute GVHD can also affect the stomach and intestines, in which case cramping, nausea, and diarrhea are present. Yellowing of the skin and eyes (jaundice) indicates that acute GVHD has affected the liver. Chronic GVHD is ranked based on its severity: stage/grade 1 is mild; stage/grade 4 is severe. Chronic GVHD develops three months or later following transplantation. The symptoms of chronic GVHD are similar to those of acute GVHD, but in addition, chronic GVHD may also affect the mucous glands in the eyes, salivary glands in the mouth, and glands that lubricate the stomach lining and intestines. Any of the populations of immune cells disclosed herein can be utilized. Examples of a transplanted organ include a solid organ transplant, such as kidney, liver, skin, pancreas, lung and/or heart, or a cellular transplant such as islets, hepatocytes, myoblasts, bone marrow, or hematopoietic or other stem cells. The transplant can be a composite transplant, such as tissues of the face. Immune cells can be administered prior to transplantation, concurrently with transplantation, or following transplantation. In some embodiments, the immune cells are administered prior to the transplant, such as at least 1 hour, at least 12 hours, at least 1 day, at least 2 days, at least 3 days, at least 4 days, at least 5 days, at least 6 days, at least 1 week, at least 2 weeks, at least 3 weeks, at least 4 weeks, or at least 1 month prior to the transplant. In one specific, non-limiting example, administration of the therapeutically effective amount of immune cells occurs 3-5 days prior to transplantation.

In some embodiments, the subject can be administered nonmyeloablative lymphodepleting chemotherapy prior to the immune cell therapy. The nonmyeloablative lymphodepleting chemotherapy can be any suitable such therapy, which can be administered by any suitable route. The nonmyeloablative lymphodepleting chemotherapy can comprise, for example, the administration of cyclophosphamide and fludarabine, particularly if the cancer is melanoma, which can be metastatic. An exemplary route of administering cyclophosphamide and fludarabine is intravenously. Likewise, any suitable dose of cyclophosphamide and fludarabine can be administered. In particular aspects, around 60 mg/kg of cyclophosphamide is administered for two days after which around 25 mg/m.sup.2 fludarabine is administered for five days.

In certain embodiments, a growth factor that promotes the growth and activation of the immune cells is administered to the subject either concomitantly with the immune cells or subsequently to the immune cells. The immune cell growth factor can be any suitable growth factor that promotes the growth and activation of the immune cells. Examples of suitable immune cell growth factors include IL-2, IL-7, IL-15, and IL-12, which can be used alone or in various combinations, such as IL-2 and IL-7, IL-2 and IL-15, IL-7 and IL-15, IL-2, IL-7 and IL-15, IL-12 and IL-7, IL-12 and IL-15, or IL-12 and IL2.

Therapeutically effective amounts of immune cells can be administered by a number of routes, including parenteral administration, for example, intravenous, intraperitoneal, intramuscular, intrasternal, or intraarticular injection, or infusion.

The therapeutically effective amount of immune cells for use in adoptive cell therapy is that amount that achieves a desired effect in a subject being treated. For instance, this can be the amount of immune cells necessary to inhibit advancement, or to cause regression of an autoimmune or alloimmune disease, or which is capable of relieving symptoms caused by an autoimmune disease, such as pain and inflammation. It can be the amount necessary to relieve symptoms associated with inflammation, such as pain, edema and elevated temperature. It can also be the amount necessary to diminish or prevent rejection of a transplanted organ.

The immune cell population can be administered in treatment regimens consistent with the disease, for example a single or a few doses over one to several days to ameliorate a disease state or periodic doses over an extended time to inhibit disease progression and prevent disease recurrence. The precise dose to be employed in the formulation will also depend on the route of administration, and the seriousness of the disease or disorder, and should be decided according to the judgment of the practitioner and each patient's circumstances. The therapeutically effective amount of immune cells will be dependent on the subject being treated, the severity and type of the affliction, and the manner of administration. In some embodiments, doses that could be used in the treatment of human subjects range from at least 3.8×10⁴, at least 3.8×10⁵, at least 3.8×10⁶, at least 3.8×10⁷, at least 3.8×10⁸, at least 3.8×10⁹, or at least 3.8×10¹⁰ immune cells/m². In a certain embodiment, the dose used in the treatment of human subjects ranges from about 3.8×10⁹ to about 3.8×10¹⁰ immune cells/m². In additional embodiments, a therapeutically effective amount of immune cells can vary from about 5×10⁶ cells per kg body weight to about 7.5×10⁸ cells per kg body weight, such as about 2×10⁷ cells to about 5×10⁸ cells per kg body weight, or about 5×10⁷ cells to about 2×10⁸ cells per kg body weight. The exact amount of immune cells is readily determined by one of skill in the art based on the age, weight, sex, and physiological condition of the subject. Effective doses can be extrapolated from dose-response curves derived from in vitro or animal model test systems.

The immune cells may be administered in combination with one or more other therapeutic agents for the treatment of the immune-mediated disorder. Combination therapies can include, but are not limited to, one or more anti-microbial agents (for example, antibiotics, anti-viral agents and anti-fungal agents), anti-tumor agents (for example, fluorouracil, methotrexate, paclitaxel, fludarabine, etoposide, doxorubicin, or vincristine), immune-depleting agents (for example, fludarabine, etoposide, doxorubicin, or vincristine), immunosuppressive agents (for example, azathioprine, or glucocorticoids, such as dexamethasone or prednisone), anti-inflammatory agents (for example, glucocorticoids such as hydrocortisone, dexamethasone or prednisone, or non-steroidal anti-inflammatory agents such as acetylsalicylic acid, ibuprofen or naproxen sodium), cytokines (for example, interleukin-10 or transforming growth factor-beta), hormones (for example, estrogen), or a vaccine. In addition, immunosuppressive or tolerogenic agents including but not limited to calcineurin inhibitors (e.g., cyclosporin and tacrolimus); mTOR inhibitors (e.g., Rapamycin); mycophenolate mofetil, antibodies (e.g., recognizing CD3, CD4, CD40, CD154, CD45, IVIG, or B cells); chemotherapeutic agents (e.g., Methotrexate, Treosulfan, Busulfan); irradiation; or chemokines, interleukins or their inhibitors (e.g., BAFF, IL-2, anti-IL-2R, IL-4, JAK kinase inhibitors) can be administered. Such additional pharmaceutical agents can be administered before, during, or after administration of the immune cells, depending on the desired effect. This administration of the cells and the agent can be by the same route or by different routes, and either at the same site or at a different site.

In certain embodiments, the compositions and methods of the present embodiments involve an immune cell population in combination with at least one additional therapy. The additional therapy may be radiation therapy, surgery (e.g., lumpectomy and a mastectomy), chemotherapy, gene therapy, DNA therapy, viral therapy, RNA therapy, immunotherapy, bone marrow transplantation, nanotherapy, monoclonal antibody therapy, or a combination of the foregoing. The additional therapy may be in the form of adjuvant or neoadjuvant therapy.

In some embodiments, the additional therapy is the administration of small molecule enzymatic inhibitor or anti-metastatic agent. In some embodiments, the additional therapy is the administration of side-effect limiting agents (e.g., agents intended to lessen the occurrence and/or severity of side effects of treatment, such as anti-nausea agents, etc.). In some embodiments, the additional therapy is radiation therapy. In some embodiments, the additional therapy is surgery. In some embodiments, the additional therapy is a combination of radiation therapy and surgery. In some embodiments, the additional therapy is gamma irradiation. In some embodiments, the additional therapy is therapy targeting PBK/AKT/mTOR pathway, HSP90 inhibitor, tubulin inhibitor, apoptosis inhibitor, and/or chemopreventative agent. The additional therapy may be one or more of the chemotherapeutic agents known in the art.

An immune cell therapy may be administered before, during, after, or in various combinations relative to an additional cancer therapy, such as immune checkpoint therapy. The administrations may be in intervals ranging from concurrently to minutes to days to weeks. In embodiments where the immune cell therapy is provided to a patient separately from an additional therapeutic agent, one would generally ensure that a significant period of time did not expire between the time of each delivery, such that the two compounds would still be able to exert an advantageously combined effect on the patient. In such instances, it is contemplated that one may provide a patient with the antibody therapy and the anti-cancer therapy within about 12 to 24 or 72 h of each other and, more particularly, within about 6-12 h of each other. In some situations it may be desirable to extend the time period for treatment significantly where several days (2, 3, 4, 5, 6, or 7) to several weeks (1, 2, 3, 4, 5, 6, 7, or 8) lapse between respective administrations.

Administration of any compound or therapy of the present embodiments to a patient will follow general protocols for the administration of such compounds, taking into account the toxicity, if any, of the agents. Therefore, in some embodiments there is a step of monitoring toxicity that is attributable to combination therapy.

A wide variety of chemotherapeutic agents may be used in conjunction with the produced immune cells. The term “chemotherapy” refers to the use of drugs to treat cancer. A “chemotherapeutic agent” is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis.

Examples of chemotherapeutic agents include alkylating agents, such as thiotepa and cyclosphosphamide; alkyl sulfonates, such as busulfan, improsulfan, and piposulfan; aziridines, such as benzodopa, carboquone, meturedopa, and uredopa; ethylenimines and methylamelamines, including altretamine, triethylenemelamine, trietylenephosphoramide, triethiylenethiophosphoramide, and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards, such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, and uracil mustard; nitrosureas, such as carmustine, chlorozotocin, fotemustine, lomustine, nimustine, and ranimnustine; antibiotics, such as the enediyne antibiotics (e.g., calicheamicin, especially calicheamicin gammalI and calicheamicin omegaIl); dynemicin, including dynemicin A; bisphosphonates, such as clodronate; an esperamicin; as well as neocarzinostatin chromophore and related chromoprotein enediyne antiobiotic chromophores, aclacinomysins, actinomycin, authramycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins, such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, and zorubicin; anti-metabolites, such as methotrexate and 5-fluorouracil (5-FU); folic acid analogues, such as denopterin, pteropterin, and trimetrexate; purine analogs, such as fludarabine, 6-mercaptopurine, thiamiprine, and thioguanine; pyrimidine analogs, such as ancitabine, azacitidine, 6-azauridine, carmofur, cytarabine, dideoxyuridine, doxifluridine, enocitabine, and floxuridine; androgens, such as calusterone, dromostanolone propionate, epitiostanol, mepitiostane, and testolactone; anti-adrenals, such as mitotane and trilostane; folic acid replenisher, such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids, such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSKpolysaccharide complex; razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; taxoids, e.g., paclitaxel and docetaxel gemcitabine; 6-thioguanine; mercaptopurine; platinum coordination complexes, such as cisplatin, oxaliplatin, and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluorometlhylornithine (DMFO); retinoids, such as retinoic acid; capecitabine; carboplatin, procarbazine, plicomycin, gemcitabien, navelbine, farnesyl-protein tansferase inhibitors, transplatinum, and pharmaceutically acceptable salts, acids, or derivatives of any of the above.

In some embodiments, radiotherapy it provided to the individual in addition to the immune cells produced herein. The radiation may include gamma-rays, X-rays, and/or the directed delivery of radioisotopes to tumor cells. Other forms of DNA damaging factors are also contemplated, such as microwaves, proton beam irradiation (U.S. Pat. Nos. 5,760,395 and 4,870,287), and UV-irradiation. It is most likely that all of these factors affect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

The skilled artisan will also understand that additional immunotherapies may be used in combination or in conjunction with the immune cells produced by method encompassed herein. In the context of cancer treatment, immunotherapeutics, generally, rely on the use of immune effector cells and molecules to target and destroy cancer cells. Rituximab is such an example. The immune effector may be, for example, an antibody specific for some marker on the surface of a tumor cell. The antibody alone may serve as an effector of therapy or it may recruit other cells to actually affect cell killing. The antibody also may be conjugated to a drug or toxin (chemotherapeutic, radionuclide, ricin A chain, cholera toxin, pertussis toxin, etc.) and serve as a targeting agent. Alternatively, the effector may be a lymphocyte carrying a surface molecule that interacts, either directly or indirectly, with a tumor cell target. Various effector cells include cytotoxic T cells and NK cells

Antibody-drug conjugates have emerged as a breakthrough approach to the development of cancer therapeutics. Cancer is one of the leading causes of deaths in the world. Antibody-drug conjugates (ADCs) comprise monoclonal antibodies (MAbs) that are covalently linked to cell-killing drugs. This approach combines the high specificity of MAbs against their antigen targets with highly potent cytotoxic drugs, resulting in “armed” MAbs that deliver the payload (drug) to tumor cells with enriched levels of the antigen. Targeted delivery of the drug also minimizes its exposure in normal tissues, resulting in decreased toxicity and improved therapeutic index. The approval of two ADC drugs, ADCETRIS® (brentuximab vedotin) in 2011 and KADCYLA® (trastuzumab emtansine or T-DM1) in 2013 by FDA validated the approach. There are currently more than 30 ADC drug candidates in various stages of clinical trials for cancer treatment (Leal et al., 2014). As antibody engineering and linker-payload optimization are becoming more and more mature, the discovery and development of new ADCs are increasingly dependent on the identification and validation of new targets that are suitable to this approach and the generation of targeting MAbs. Two criteria for ADC targets are upregulated/high levels of expression in tumor cells and robust internalization.

In one aspect of immunotherapy, the tumor cell must bear some marker that is amenable to targeting, i.e., is not present on the majority of other cells. Many tumor markers exist and any of these may be suitable for targeting in the context of the present embodiments. Common tumor markers include CD20, carcinoembryonic antigen, tyrosinase (p97), gp68, TAG-72, HMFG, Sialyl Lewis Antigen, MucA, MucB, PLAP, laminin receptor, erb B, and p155. An alternative aspect of immunotherapy is to combine anticancer effects with immune stimulatory effects. Immune stimulating molecules also exist including: cytokines, such as IL-2, IL-4, IL-12, GM-CSF, gamma-IFN, chemokines, such as MIP-1, MCP-1, IL-8, and growth factors, such as FLT3 ligand.

Examples of immunotherapies currently under investigation or in use are immune adjuvants, e.g., Mycobacterium bovis, Plasmodium falciparum, dinitrochlorobenzene, and aromatic compounds (U.S. Pat. Nos. 5,801,005 and 5,739,169; Hui and Hashimoto, 1998; Christodoulides et al., 1998); cytokine therapy, e.g., interferons .alpha., .beta., and .gamma., IL-1, GM-CSF, and TNF (Bukowski et al., 1998; Davidson et al., 1998; Hellstrand et al., 1998); gene therapy, e.g., TNF, IL-1, IL-2, and p53 (Qin et al., 1998; Austin-Ward and Villaseca, 1998; U.S. Pat. Nos. 5,830,880 and 5,846,945); and monoclonal antibodies, e.g., anti-CD20, anti-ganglioside GM2, and anti-p185 (Hollander, 2012; Hanibuchi et al., 1998; U.S. Pat. No. 5,824,311). It is contemplated that one or more anti-cancer therapies may be employed with the antibody therapies described herein.

In some embodiments, the immunotherapy may be an immune checkpoint inhibitor. Immune checkpoints either turn up a signal (e.g., co-stimulatory molecules) or turn down a signal. Inhibitory immune checkpoints that may be targeted by immune checkpoint blockade include adenosine A2A receptor (A2AR), B7-H3 (also known as CD276), B and T lymphocyte attenuator (BTLA), cytotoxic T-lymphocyte-associated protein 4 (CTLA-4, also known as CD152), indoleamine 2,3-dioxygenase (IDO), killer-cell immunoglobulin (KIR), lymphocyte activation gene-3 (LAG3), programmed death 1 (PD-1), T-cell immunoglobulin domain and mucin domain 3 (TIM-3) and V-domain Ig suppressor of T cell activation (VISTA). In particular, the immune checkpoint inhibitors target the PD-1 axis and/or CTLA-4.

The immune checkpoint inhibitors may be drugs such as small molecules, recombinant forms of ligand or receptors, or, in particular, are antibodies, such as human antibodies (e.g., International Patent Publication WO2015016718; Pardoll, Nat Rev Cancer, 12(4): 252-64, 2012; both incorporated herein by reference). Known inhibitors of the immune checkpoint proteins or analogs thereof may be used, in particular chimerized, humanized or human forms of antibodies may be used. As the skilled person will know, alternative and/or equivalent names may be in use for certain antibodies mentioned in the present disclosure. Such alternative and/or equivalent names are interchangeable in the context of the present disclosure. For example it is known that lambrolizumab is also known under the alternative and equivalent names MK-3475 and pembrolizumab.

In some cases, surgery is performed for an individual that will receive the immune cells of the disclosure or that have received them. Approximately 60% of persons with cancer will undergo surgery of some type, which includes preventative, diagnostic or staging, curative, and palliative surgery. Curative surgery includes resection in which all or part of cancerous tissue is physically removed, excised, and/or destroyed and may be used in conjunction with other therapies, such as the treatment of the present embodiments, chemotherapy, radiotherapy, hormonal therapy, gene therapy, immunotherapy, and/or alternative therapies. Tumor resection refers to physical removal of at least part of a tumor. In addition to tumor resection, treatment by surgery includes laser surgery, cryosurgery, electrosurgery, and microscopically-controlled surgery (Mohs' surgery). Upon excision of part or all of cancerous cells, tissue, or tumor, a cavity may be formed in the body. Treatment may be accomplished by perfusion, direct injection, or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

It is contemplated that other agents may be used in combination with certain aspects of the present embodiments to improve the therapeutic efficacy of treatment. These additional agents include agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Increases in intercellular signaling by elevating the number of GAP junctions would increase the anti-hyperproliferative effects on the neighboring hyperproliferative cell population. In other embodiments, cytostatic or differentiation agents can be used in combination with certain aspects of the present embodiments to improve the anti-hyperproliferative efficacy of the treatments. Inhibitors of cell adhesion are contemplated to improve the efficacy of the present embodiments. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with certain aspects of the present embodiments to improve the treatment efficacy.

### VI. Articles of Manufacture or Kits

An article of manufacture or a kit is provided comprising immune cells produced from selected cord blood unit(s) is also provided herein. The article of manufacture or kit can further comprise a package insert comprising instructions for using the immune cells to treat or delay progression of cancer in an individual or to enhance immune function of an individual having cancer. Any of the antigen-specific immune cells described herein may be included in the article of manufacture or kits. Suitable containers include, for example, bottles, vials, bags and syringes. The container may be formed from a variety of materials such as glass, plastic (such as polyvinyl chloride or polyolefin), or metal alloy (such as stainless steel or hastelloy). In some embodiments, the container holds the formulation and the label on, or associated with, the container may indicate directions for use. The article of manufacture or kit may further include other materials desirable from a commercial and user standpoint, including other buffers, diluents, filters, needles, syringes, and package inserts with instructions for use. In some embodiments, the article of manufacture further includes one or more of another agent (e.g., a chemotherapeutic agent, and anti-neoplastic agent). Suitable containers for the one or more agent include, for example, bottles, vials, bags and syringes.

In specific embodiments the article of manufacture comprises cryopreserved immune cells produced by methods described herein. The cryopreserved cells may be frozen with a particular cryoprotectant suited to prevent them from damage upon freezing or thawing.

### EXAMPLES

The following examples are included to demonstrate particular embodiments of the disclosure. It should be appreciated by those of skill in the art that the techniques disclosed in the examples that follow represent techniques discovered by the inventors to function well in the practice of the embodiments of the disclosure, and thus can be considered to constitute particular modes for its practice. However, those of skill in the art should, in light of the present disclosure, appreciate that many changes can be made in the specific embodiments that are disclosed and still obtain a like or similar result without departing from the spirit and scope of the disclosure.

### Example 1

### Pre-Freezing Cbu Characteristics Predict Clinical Response

Studies in the present example characterize whether pre-freezing cord blood unit (CBU) characteristics can be used to identify those CBU that are more likely to result in a clinically efficacious cell products.

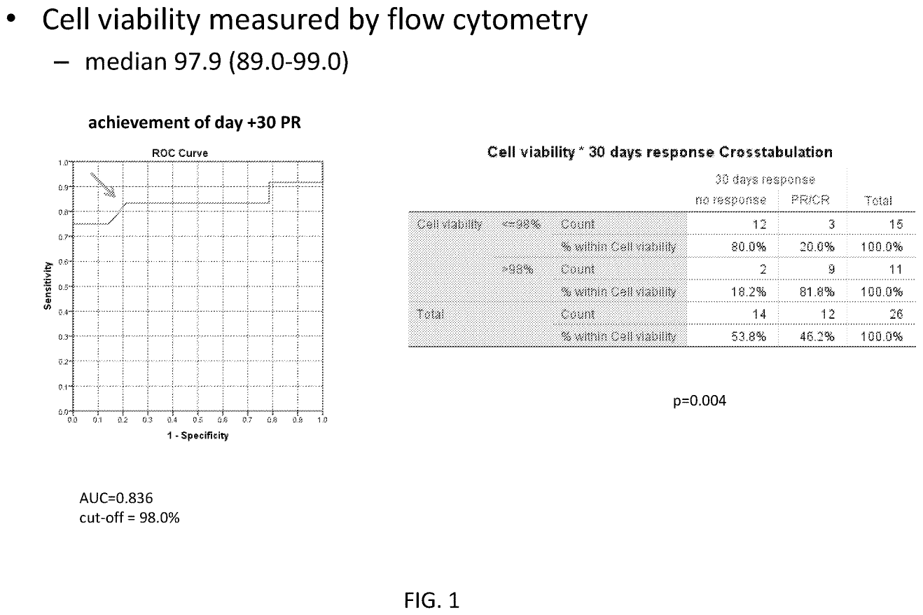

The inventors utilized an operating receiver characteristic (ROC) curve to characterize the predictive value of the CBU characteristic of interest and identify the appropriate cut-off value that allows classification of each individual CBU as likely (“good”) or unlikely (“bad”) to induce clinical response in patients. For example, in FIG. 1 the CBU cell viability was examined. The arrow on the ROC curve indicates the value on the CBU cell viability that can be used to classify the CBU as “good or bad” with the best sensitivity and specificity (this is determined by the closest point to 100% sensitivity and 100% sensitivity[l-specificity=0]). In this case, the value is 98%. Then, the response that patient had to CAR-NK cells was examined. Patients who received CAR-NK cells produced from CBU with a viability >98% had 81.8% response, but patients who received CAR-NK cells manufactured from CBUs with a viability <98% had only 20% response. This result is statistically significant (Fisher exact test, p=0.004). Then, a logistic regression model was utilized to verify that this result is independent of the clinical characteristics of the patient, such us remission status.

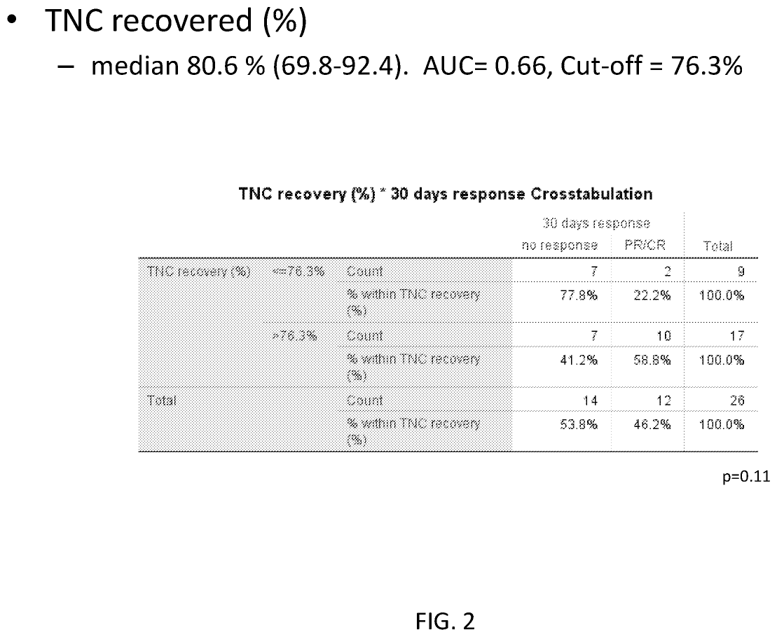

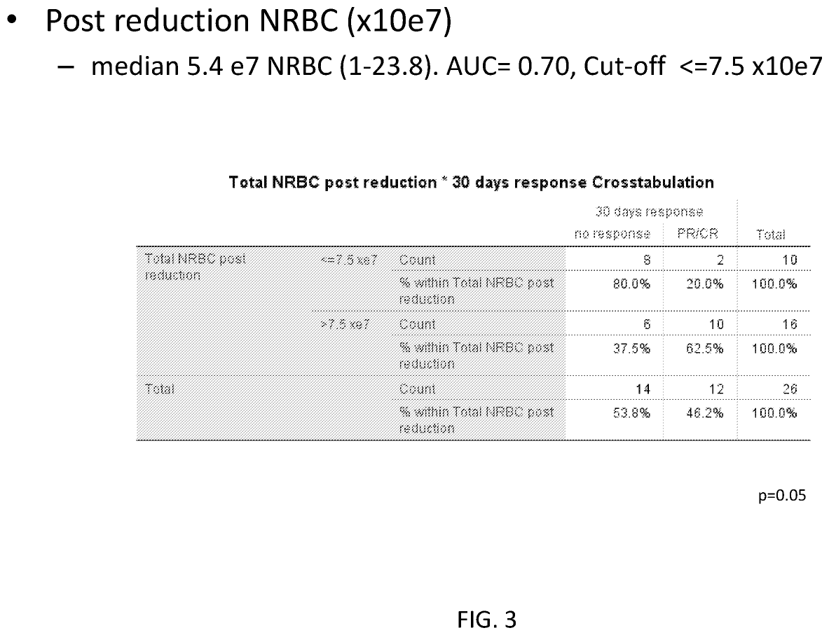

This methodology described above was applied to investigate other variables, such as total mononuclear cell (TNC) recovery. The optimal cut-off for the prediction of responses is 76.3% (FIG. 2). In this case, the difference in responses between patients who received CAR-NK cells manufactured from CBUs with a TNC higher and lower than 73.6 (58.8% vs. 22.2%) is not statistically significant (p=0.11). However, a multivariate logistic regression models shows that the influence of TNC on outcome is statistically significant when the effect of confounding clinical variables (such as remission status) is taken into account.

This methodology was also employed as above with other CBU characteristics. In one case, the nucleated red blood cell (NRBC) content of the CBU was characterized. Patients treated with CAR-NK manufactured from CBUs with a low cell content (<7.5×10e7 NRBC) have a higher response rate than patients treated with CAR-NK cells manufactured from CBU with higher NRBC content (62.5% vs. 20% p=0.05). Again, a multivariate logistic model was employed to demonstrate that this effect is independent of clinical variables.

### Example 2

### Pre-Freezing CBU Characteristics can be Combined to Identify “Super-Cbus”

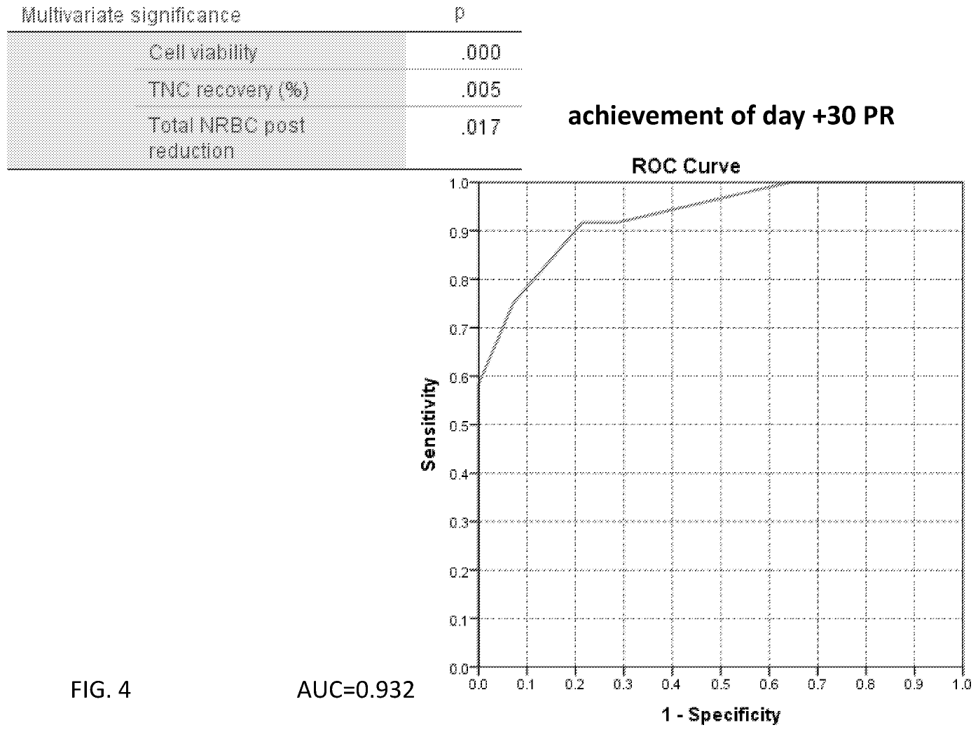

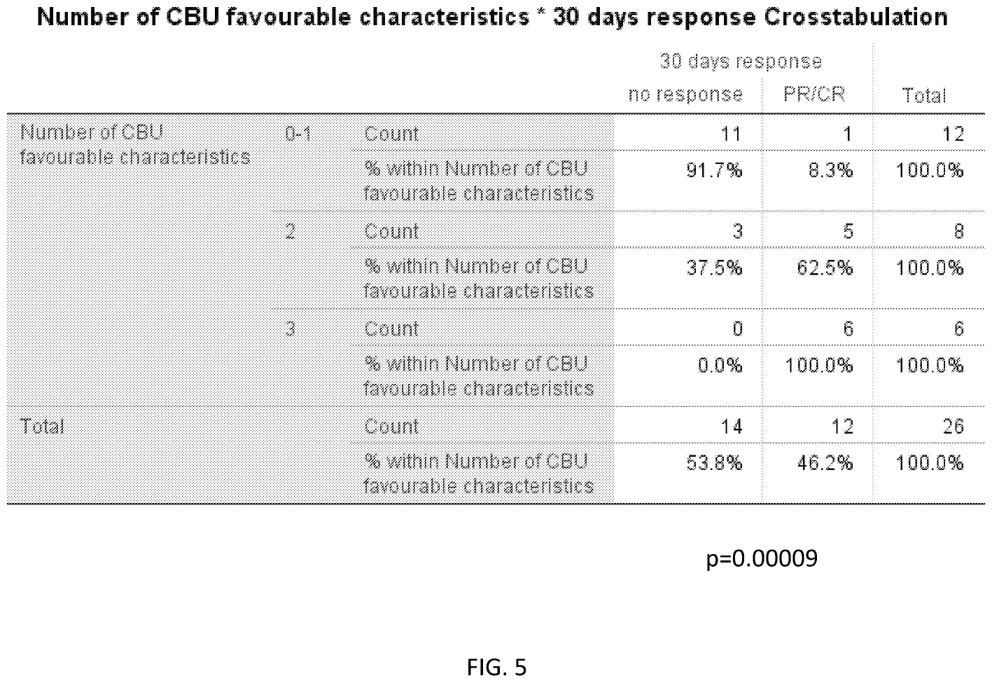

The three CBU characteristics described in Example 1 are independent predictors for response in a logistic multivariate model adjusted for clinical characteristics. For this reason, the three can be combined to define the criteria for an “optimal CBU”. FIG. 4 shows the multivariate statistical significance for the three CBU characteristics referred to in Example 1. Then, a ROC curve (right panel) was utilized to measure the predictive value of the CBU criteria on response to the infused CAR-NK cell product. The area under the curve (AUC) of 0.932 indicates that meeting the three criteria (viability >98%, TNC recovery >76.3% and NRBC content <7.5×10e7) is an excellent predictor for response. FIG. 5 shows the responses of patients treated with CAR-NK cells manufactured from CBU units that meet the three criteria referred to here and in Example 1 (100%) two criteria (62.5%) and less than 2 criteria (8.3%). This differences are statistically significant (p=0.00009). The number of favorable cord characteristics is the only independent predictor for response in a multivariate model including clinical characteristics.

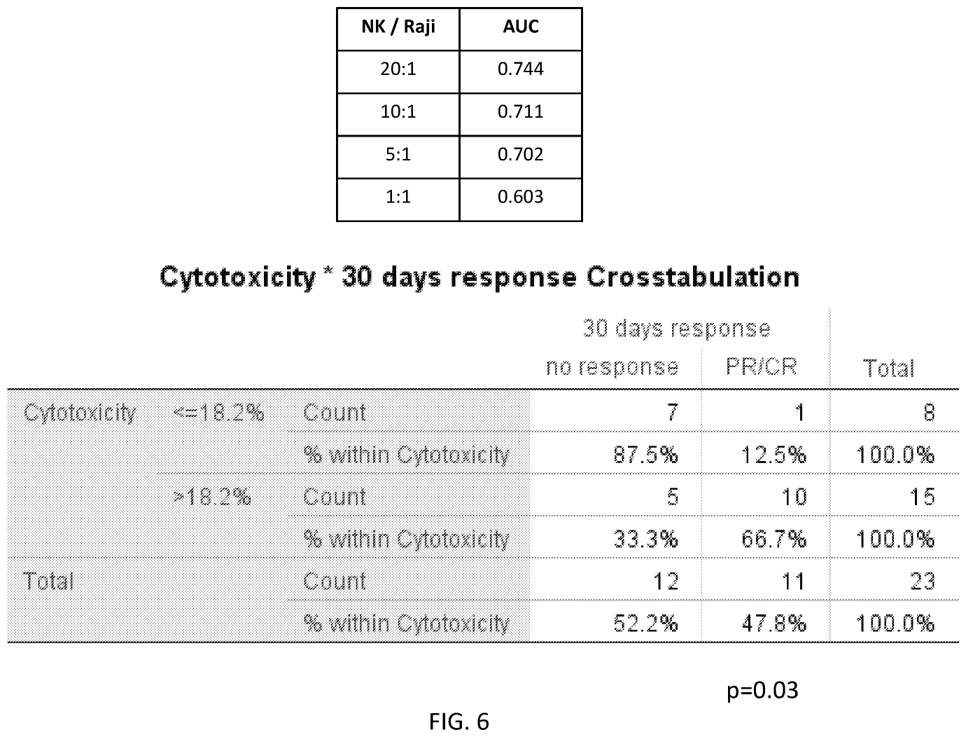

The predictive value of other CBU-associated variables were characterized that would not be known upon cord selection but that could be elucidated during the manufacture of the cell products. In specific embodiments, such variables would be determined post-thaw. This can be utilized to disregard cell products after manufacture if they do not meet the appropriate criteria. In FIG. 6, it was examined whether the cytotoxicity of NK cells obtained from the frozen CBUs can predict the clinical response to CAR-NK cells. Using the methodology described above, it was shown that patients treated with CAR-NK cells produced from CBUs that have a NK cell cytotoxicity against Raji cell line at a ratio of 20:1 >18.2 have a higher response rate than patients treated with CAR-NK cell derived from CBUs with lower cytotoxicity (66.7% vs. 12.5% p=0.03). Again, a multivariate logistic model was used to demonstrate that this effect is independent of other variables.

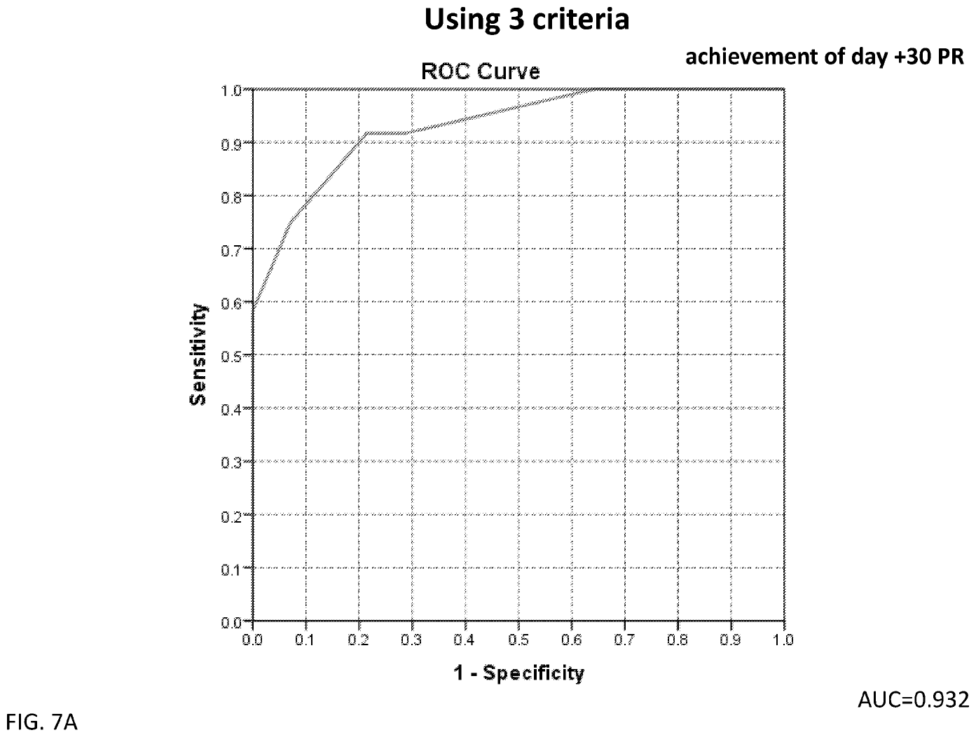

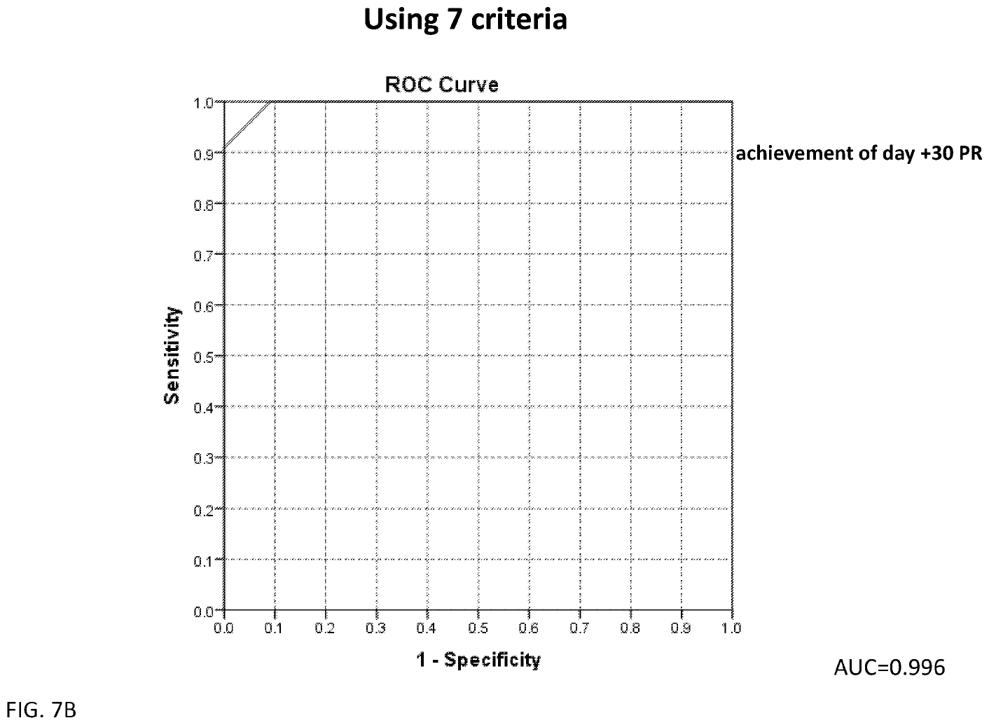

In particular embodiments, other variables may be considered to improve prediction for clinical response. Examples include the following: (1) gestational age of the fetus or infant from which the cord blood was obtained is <39 weeks; (2) post-thaw viability of cord blood cells is >86.5%; (3) NK cell expansion between days 0 and 6 in culture is greater than or equal to 7-fold; and/or (4) NK cell expansion between days 6 and 15 in culture is greater than or equal to 10⁵-fold. FIGS. 7A-7B demonstrate the predictive value of the three criteria set (viability >98%, TNC recovery >76.3% and NRBC content <7.5×10⁷)(FIG. 7A) having a clinical response of 93.2%. This can be increased to 99.6% by adding the four variables described immediately above (FIG. 7B).

### Example 3

### A Method for Selection of Cryopreserved Cord Blood Units for the Manufacture of Engineered Natural Killer Cells with the Highest Potency Against Cancer

The present example concerns identification of predictors for response for therapy that considers criteria related to selection of suitable cord blood units (CBU). The inventors investigated whether pre-freezing CBU characteristics can be used to identify those CBU that are more likely to result in a clinically efficacious cell products. The product characteristics may include pre-freezing CBU characteristics (selecting the best CBUs to produce the cell product) and/or may include post-thaw and on-production product characteristics that in specific cases may be used to reject products deemed unlikely to result in optimal responses. The present example concerns analysis based on 37 patients treated in a CD19-CAR-NK trial, with outcomes being complete response (CR) and partial response (PR)/CR at 30 days.

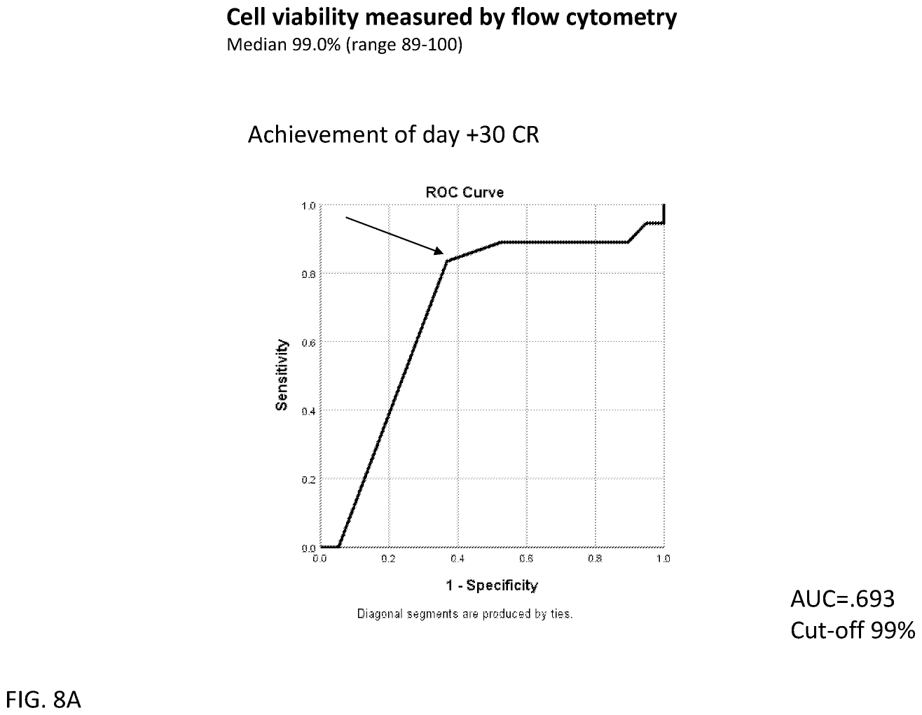

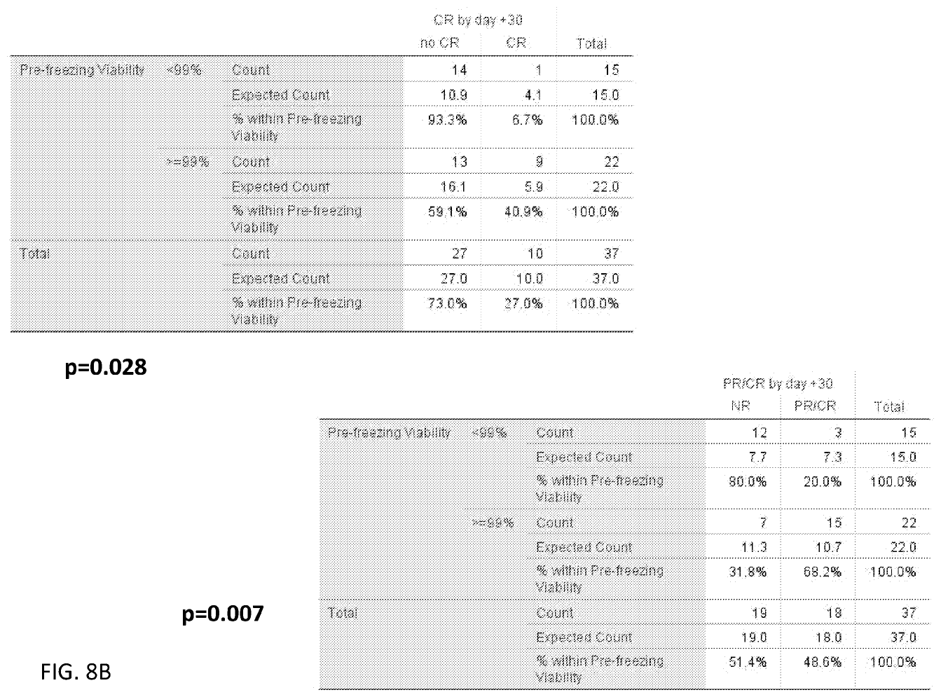

An operating receiver characteristic (ROC) curve was utilized to study the predictive value of the CBU characteristic of interest and identify the appropriate cut-off value that will allow classification of each individual CBU as likely (“good”) or unlikely (“bad”) to induce clinical response in patients. For example, in FIG. 8 the CBU cell viability was examined. The arrow on the ROC curve indicates the value on the CBU cell viability that can be used to classify the CBU as “good or bad” with the best sensitivity and specificity (this is determined by the closest point to 100% sensitivity and 100% sensitivity[1−specificity=0]). In this case the value is 99%. Then the response that patient had to CAR-NK cells was considered. Patients who received CAR-NK cells produced from CBU with a viability >99% had 40.9% CR and a 68.2% CR/PR rate. On the other hand, patients who receive CAR-NK cells manufactured from CBUs with a viability <99% had only had 6.7% CR and 20% CR/PR rates. These results are statistically significant (Fisher exact tests, p=0.028 and p=0.007 respectively). Then a logistic regression model was used to verify that this result is independent of the clinical characteristics of the patient, such us remission status.

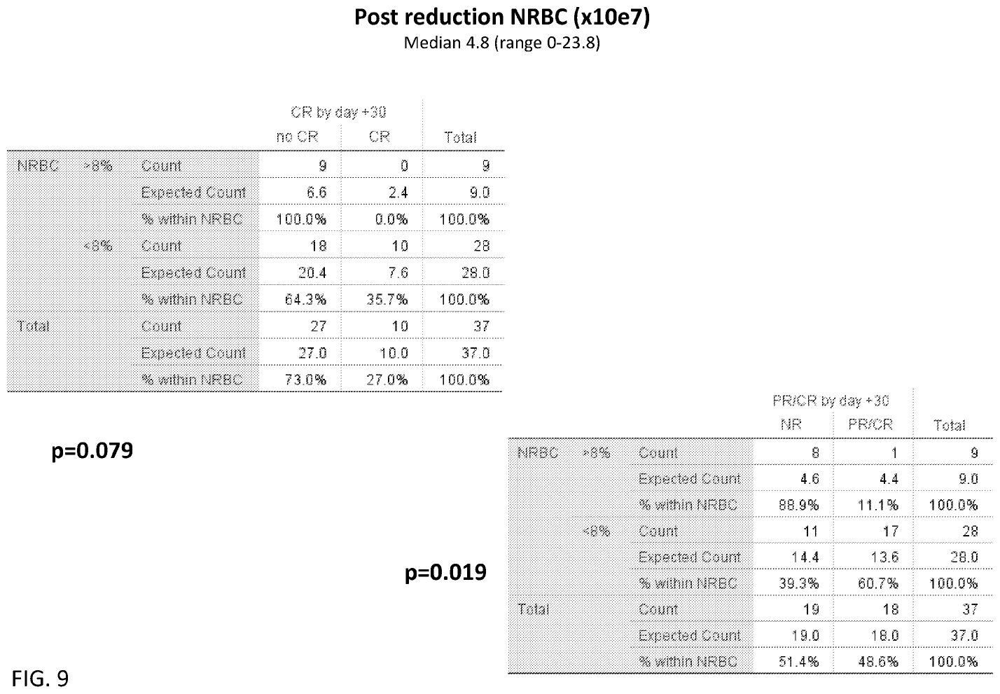

The same methodology in FIG. 8 was applied with other CBU characteristics in FIG. 9; in this case, the nucleated red blood cell (NRBC) content of the CBU was examined. Patients treated with CAR-NK manufactured from CBUs with a low cell content (<8.0 10e7 NRBC) have a higher response rate than patients treated with CAR-NK cells manufactured from CBU with higher NRBC content (35.7% vs 0% p=0.079 CR rate and 60.7% vs 11.1%, p=0.019 PR/CR rate). Again, a multivariate logistic model was used to demonstrate that this effect is independent of clinical variables.

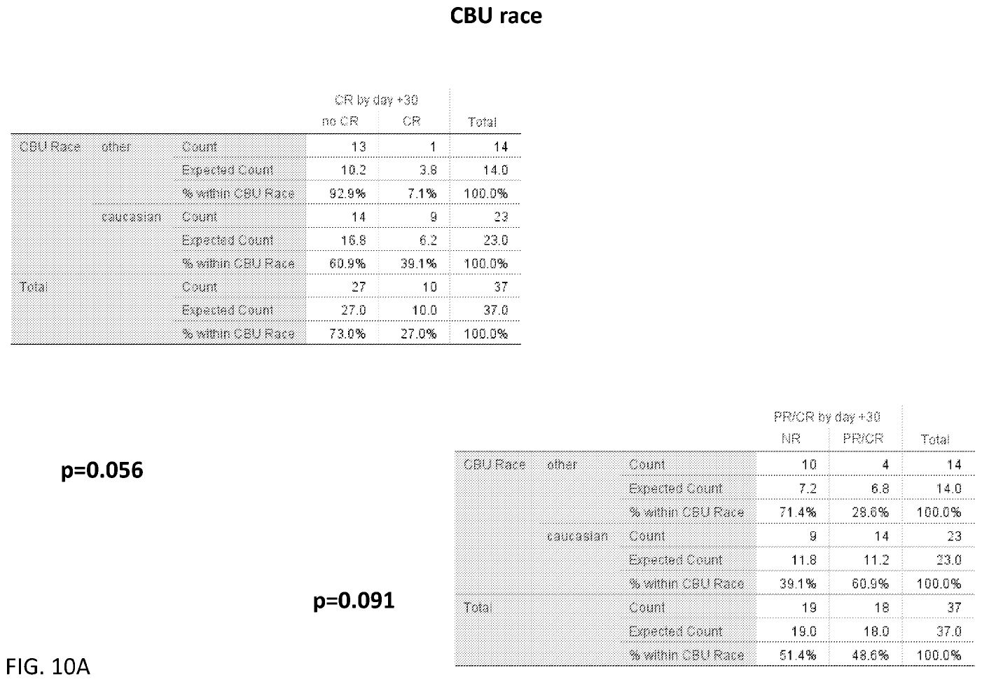

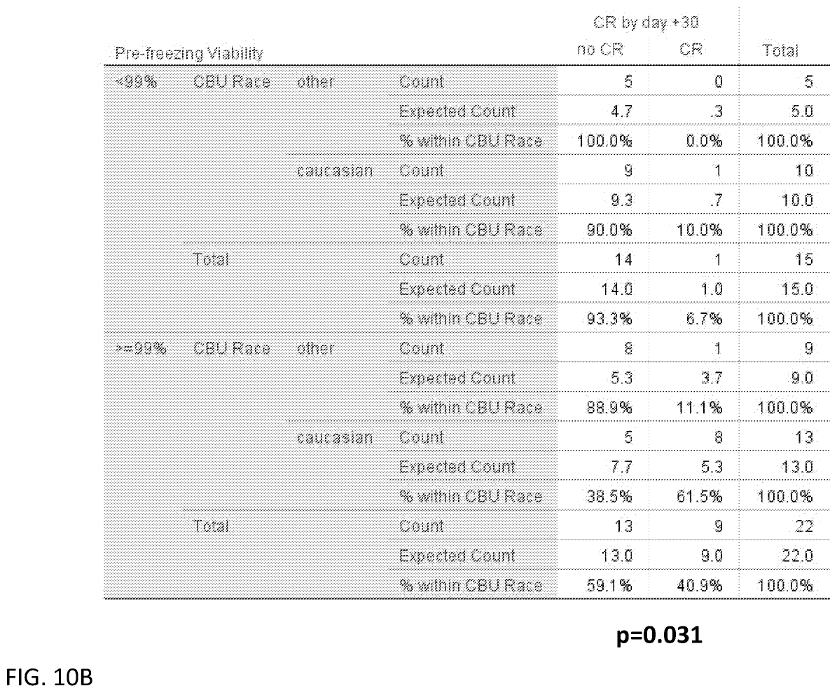

Patients treated with CAR-NK manufactured from CBUs of Caucasian race had a higher response rate than patients treated with CAR-NK cells manufactured from CBU from other ethnicities (FIG. 10A). The CBU ethnicity can be combined with other CBU characteristics to improve the selection of the CBUs that are more likely to result in clinical responses. FIG. 10B shows the result of combining the CBU race with the CBU viability. The combination of both factors increases the CR rate from 40.9% for viability alone to 61.5% when both criteria are combined (p=0.031).

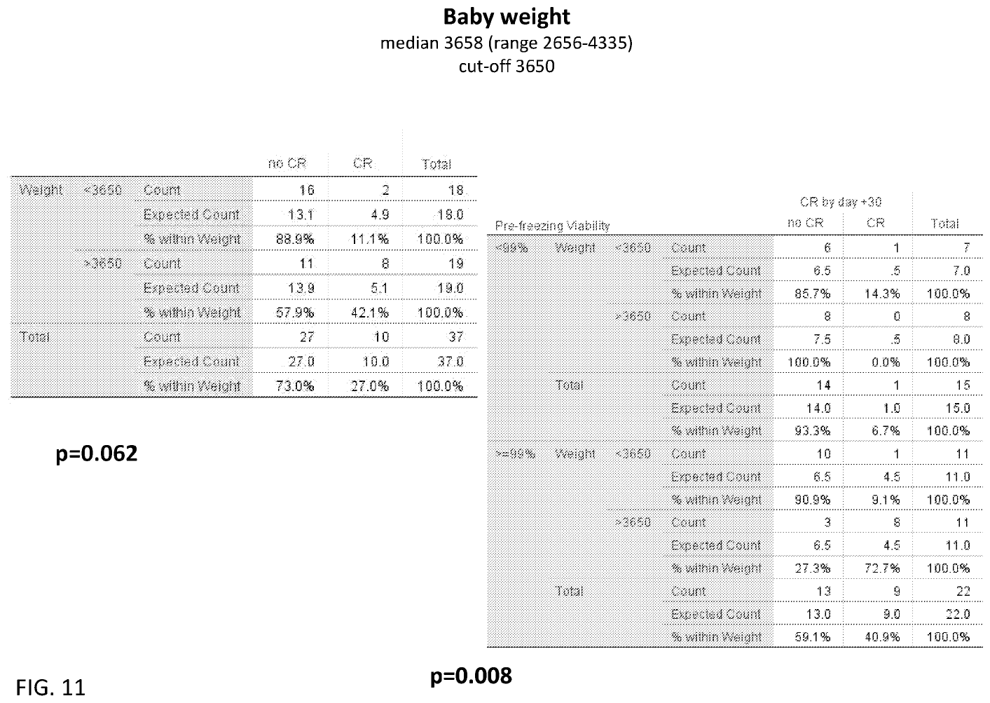

Patients treated with CAR-NK manufactured from CBUs from babies who weight >3650 grams have a higher response rate than patients treated with CAR-NK cells manufactured from CBU from smaller babies (panel in the left of FIG. 11). Like in FIG. 10, the baby weight can be combined with other CBU characteristics to better select the CBUs that are more likely to result in clinical response. The panel on the right of FIG. 11 shows the result of combining the babies weight with the CBUs viability. The combination of both factors increases the CR rate from 40.9% for viability alone to 72.7% when both criteria are combined (p=0.008).

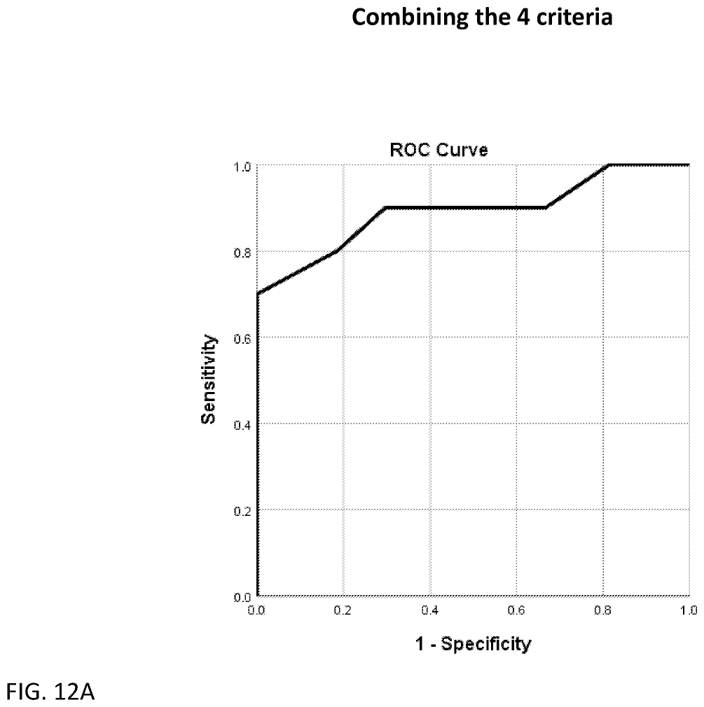

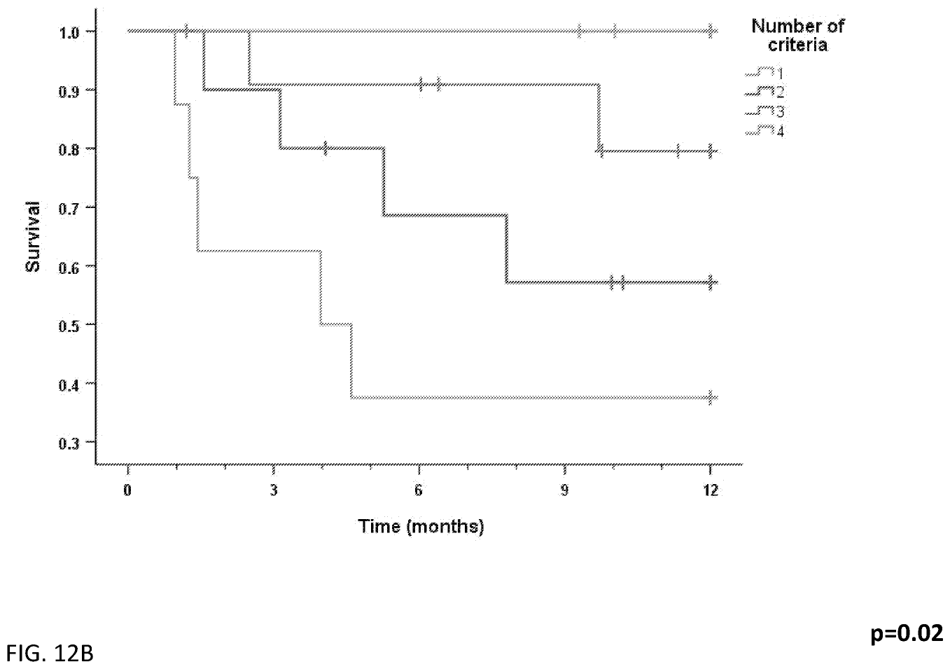

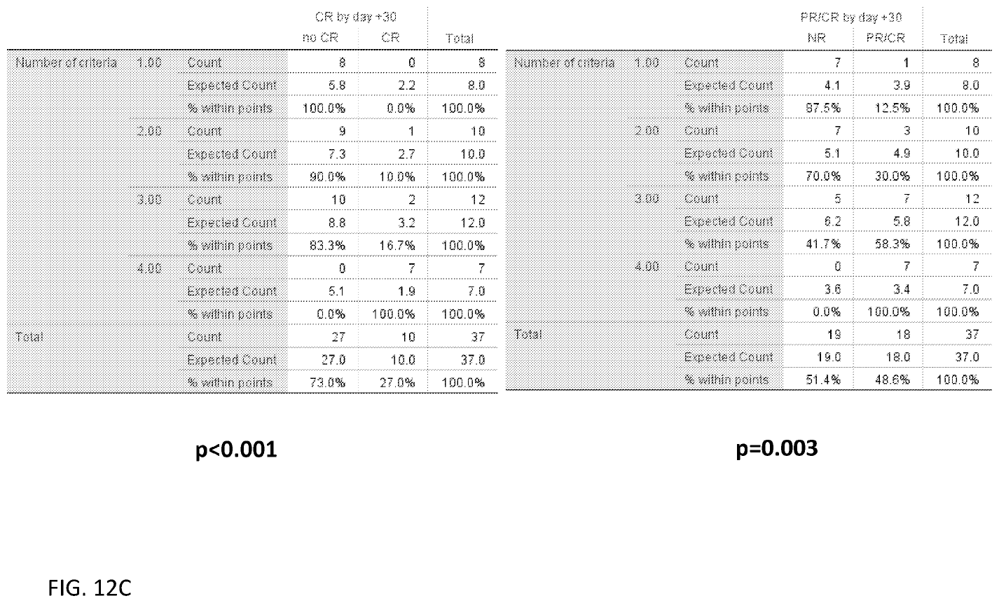

The four CBU characteristics described above in this example are independent predictors for response in a logistic multivariate model adjusted for clinic characteristics. For this reason, in some embodiments, the four can be combined to define the criteria for a “optimal CBU” (FIG. 12A). Then the inventors examined the predictive value of meeting the optimal CBU criteria on response to CAR-NK cell product using a ROC curve (FIG. 12A). The area under the curve (AUC) of 0.893 indicates that meeting the 4 three criteria (viability >99%, NRBC content <8.0, baby weight >3650 grams and Caucasian ethnicity) is an excellent predictor for response.

FIG. 12C shows the response rate (CR above panel and CR/PR below panel) according to the number of “optimal” CBU characteristics that the NK cell product that the patient received had. For example, the CR rate ranges from 0% for patients who received products derived from CBUs that only met one criteria, to 100% response rate for patients who received a cell product derived from CBUs that met the four criteria (p<0.001). Similarly, the PR/CR rates were 12.5%, 30.%, 58.3% and 100% for patients who received a cell products derived from CBUs that had 1, 2, 3 or 4 of the desired characteristics (p=0.003). FIG. 12A shows the probabilities of survival according to the number of CBU characteristics. The 12 months probability of survival for patients who received cell products derived from CBUs that had 1, 2, 3 or 4 characteristics was 37.5%, 57.1%, 79.5% and 100% respectively (p=0.02).

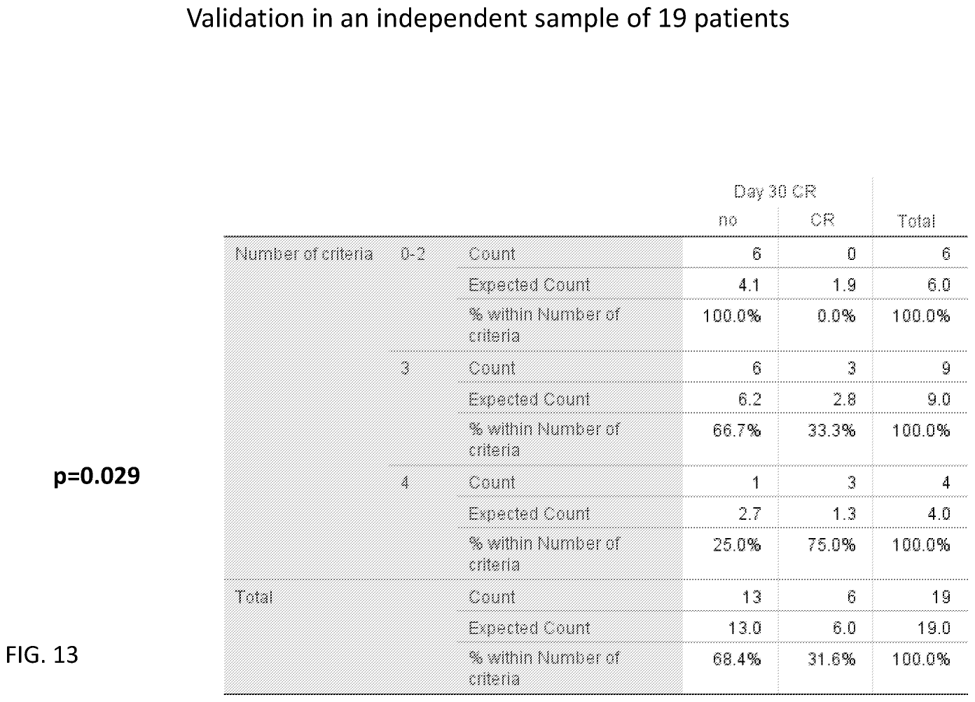

The results were validated in an independent sample of 19 patients treated with a different NK cell product with very similar results. In this case, the day +30 CR rate was 0%, 33.3%, and 75% for patients who received cell products derived from CBUs that had ≤2, 3 or 4 characteristics respectively (p=0.029) (FIG. 13).

In some embodiments, there are additional parameters to improve the prediction for clinical responses, such as gestational age ≤38 weeks; intra utero collection method; male baby; pre-process volume ≤120 ml; CD34% >0.4%; NK cell expansion between days 0 and 15 in culture ≥450 fold; and NK cell expansion between days 6 and 15 in culture ≥70 fold.

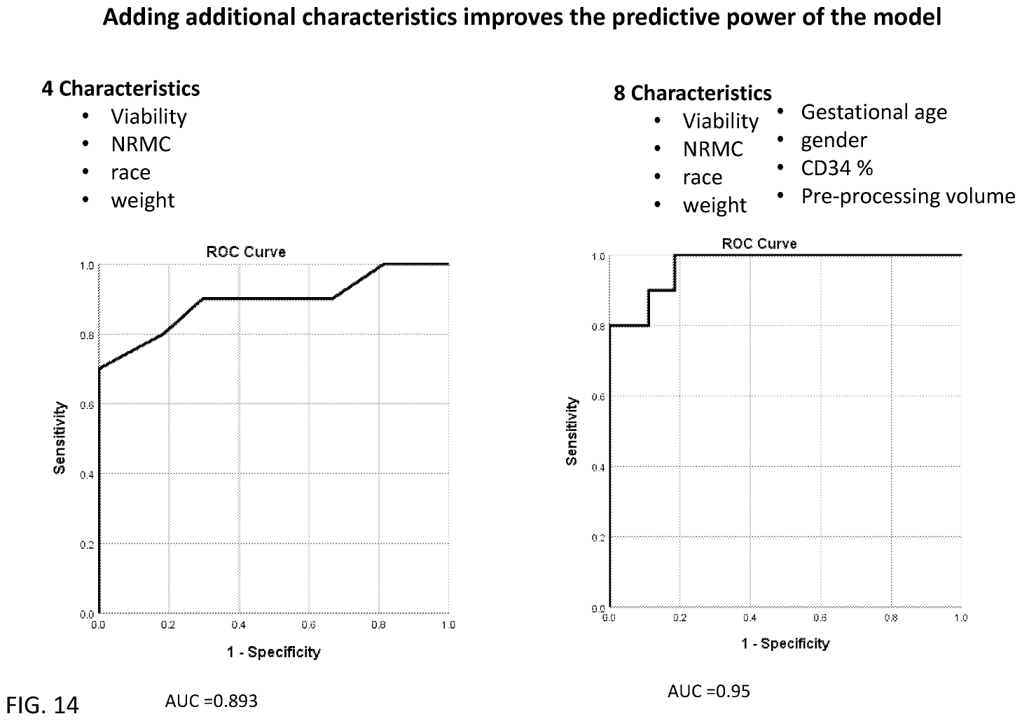

As shown before, the predictive value of the four criteria set (viability ≥99%, NRBC content <8, Caucasian ethnicity and baby's weight >3650 grams on clinical response is 89.3%. This can be increased to 97.0% by adding the variables described above (FIG. 14).

Although the present disclosure and its advantages have been described in detail, it should be understood that various changes, substitutions and alterations can be made herein without departing from the spirit and scope of the design as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the present disclosure, processes, machines, manufacture, compositions of matter, means, methods, or steps, presently existing or later to be developed that perform substantially the same function or achieve substantially the same result as the corresponding embodiments described herein may be utilized according to the present disclosure. Accordingly, the appended claims are intended to include within their scope such processes, machines, manufacture, compositions of matter, means, methods, or steps.

What is claimed is:

1. A method of selecting a cord blood composition, comprising the steps of:
measuring prior to cryopreservation of the cord blood composition or considering prior to cryopreservation:
(a) cord blood cell viability;
(b) optionally total mononuclear cell (TNC) recovery;
(c) nucleated red blood cell (NRBC) content;
(d) weight of the baby from which the cord blood is derived;
(e) race of the biological mother and/or biological father of the baby from which the cord blood is derived;
(f) optionally gestational age of the baby from which the cord blood is derived;
(g) optionally intra utero collection of the cord blood;
(h) optionally a biologically male baby from which the cord blood is derived;
(i) optionally a volume of the cord blood collected;
(j) optionally the number of cells of the extracted cord blood that are CD34+; and
measuring subsequent to cryopreservation (d) cytotoxicity of immune cells derived from the cord blood composition following thawing.

2. The method of claim 2, wherein the immune cells are natural killer (NK) cells.

3. The method of claim 2, further comprising the step of expanding the NK cells.

4. The method of claim 2 or 3, further comprising the step of modifying the NK cells.

5. The method of claim 4, wherein the NK cells are modified to express one or more non-endogenous gene products.

6. The method of claim 5, wherein the non-endogenous gene product comprises one or more non-endogenous receptors.

7. The method of claim 6, wherein the non-endogenous receptor is a chimeric receptor.

8. The method of claim 7, wherein the chimeric receptor is a chimeric antigen receptor.

9. The method of claim 6, wherein the non-endogenous receptor is a non-natural T-cell receptor.

10. The method of claim 5, wherein the non-endogenous gene product comprises one or more non-endogenous receptors, one or more cytokines, one or more chemokines, one or more enzymes, or a combination thereof.

11. The method of any one of claims 4-10, wherein the NK cells are modified to have disruption of expression of one or more endogenous genes in the NK cells.

12. A method of selecting a cord blood composition, comprising the steps of:
identifying a cord blood composition that, prior to cryopreservation, is determined to have one or more of the following:
(a) cord blood cell viability greater than or equal to about 98% or 99%;
(b) optionally total mononuclear cell (TNC) recovery is greater than or equal to 76.3%;
(c) nucleated red blood cell (NRBC) content less than or equal to about 7.5×10⁷ to about 8.0×10⁷;
(d) weight of the baby from which the cord blood is derived is greater than about 3650 grams;
(e) race of the biological mother and/or biological father of the baby from which the cord blood is derived is Caucasian;
(f) optionally gestational age of the baby from which the cord blood is derived is less than or equal to about 38 weeks;
(g) optionally intra utero collection of the cord blood;
(h) optionally a biologically male baby from which the cord blood is derived;
(i) optionally a volume of the cord blood collected plus anticoagulant being ≤about 120 mL;
(j) optionally cells of the extracted cord blood are >about 0.4% CD34+; and
optionally (k) measuring cytotoxicity of immune cells derived from the cord blood composition following thawing.

13. The method of claim 12, wherein the cord blood composition prior to cryopreservation is determined to have (a), (c), (d) and (e).

14. The method of claim 12 or 13, wherein the cord blood cell viability in (a) is greater than or equal to 99.0, 99.1, 99.2, 99.3, 99.4, 99.5, 99.6, 99.7, 99.8, or 99.9%.

15. The method of any one of claims 12-14, wherein the TNC recovery in (b) is greater than or equal to 77, 78, 79, 80, 81, 82, 83, 84, 85, 86, 87, 88, 89, 90, 91, 92, 93, 94, 95, 96, 97, 98, or 99%.

16. The method of any one of claims 12-15, wherein the NRBC content is less than or equal to 8.0×10⁷, 7.9×10⁷, 7.8×10⁷, 7.7×10⁷, 7.6×10⁷, 7.5×10⁷, 7.0×10⁷, 6.0×10⁷, 5.0×10⁷, 4.0×10⁷, 3.0×10⁷, 2.0×10⁷, 1.0×10⁷, 9.0×10⁶, 8.0×10⁶, 7.0×10⁶, 6.0×10⁶, 5.0×10⁶, 4.0×10⁶, 3.0×10⁶, 2.0×10⁶, 1.0×10⁶, 9.0×10⁵, 8.0×10⁵, 7.0×10⁵, 6.0×10⁵, 5.0×10⁵, 4.0×10⁵, 3.0×10⁵, 2.0×10⁵, 1.0×10⁵, 9.0×10⁴, 8.0×10⁴, 7.0×10⁴, 6.0×10⁴, 5.0×10⁴, 4.0×10⁴, 3.0×10⁴, 2.0×10⁴, 1.0×10⁴, 9.0×10³, 8.0×10³, 7.0×10³, 6.0×10³, 5.0×10³, 4.0×10³, 3.0×10³, 2.0×10¹, 1.0×10³, 9.0×10², 8.0×10², 7.0×10², 6.0×10², 5.0×10², 4.0×10², 3.0×10², 2.0×10², 1.0×10², or less.

17. The method of any one of claims 12-16, wherein weight of the baby from which the cord blood is derived is greater than about 3650, 3700, 3750, 3800, 3850, 3900, 3950, 4000, 4050, 4100, 4150, 4200, 4250, or 4500 grams.

18. The method of any one of claims 12-17, wherein the volume of the cord blood collected plus anticoagulant is ≤about 120, 115, 110, 100, 90, 80, 70, 60, or 50 mL.

19. The method of any one of claims 12-18, wherein cells of the extracted cord blood are >0.4, 0.5, 1, 2, 3, 4, 5, 10, 15, 20, or more % CD34+.

20. The method of any one of claims 12-19, further comprising the step of deriving immune cells from the thawed cord blood composition.

21. The method of claim 20, wherein the immune cells are NK cells, invariant NK cells, NK T cells, T cells B cells, monocytes, granulocytes, myeloid cells neutrophils, eosinophils, basophils, mast cells, monocytes, macrophages, dendritic cells, stem cells, or a mixture thereof.

22. The method of claim 20 or 21, wherein the immune cells derived from the cord blood composition following thawing are NK cells and the cytotoxicity is greater than or equal to 66.7%.

23. The method of claim 22, wherein the cytotoxicity is greater than or equal to 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99%.

24. The method of any one of the preceding claims, wherein the cord blood is derived from a fetus or infant at less than or equal to 38 weeks of gestational age.

25. The method of claim 24, wherein the cord blood is derived from a fetus or infant at less than or equal to 38, 37, 36, 35, 34, 33, 32, 31, 30, 29, 28, 27, 26, 25, or 24 weeks of gestational age.

26. The method of any one of the preceding claims, wherein the method further comprises determining viability of cord blood cells following thawing.

27. The method of claim 26, wherein the viability of cord blood cells following thawing is greater than or equal to 86.5%.

28. The method of claim 27, wherein the viability of cord blood cells following thawing is greater than or equal to 87, 88, 89, 90, 91, 92, 93, 94, 95, 96, 97, 98, or 99%.

29. The method of claim 21, wherein the immune cells are NK cells.

30. The method of claim 29, wherein the NK cells are expanded.

31. The method of claim 30, wherein the expansion of the NK cells between days 0 and 6 in culture is greater than or equal to 3-fold.

32. The method of claim 30 or 31, wherein the expansion of the NK cells between days 6 and 15 in culture is greater than or equal to 70-fold.

33. The method of claim 30, wherein the expansion of the NK cells between days 0 and 15 is greater than or equal to 450-fold.

34. The method of any one of claims 29-33, wherein the NK cells are modified.

35. The method of claim 34, wherein the NK cells are modified to express one or more non-endogenous gene products.

36. The method of claim 35, wherein the non-endogenous gene product is a non-endogenous receptor.

37. The method of claim 36, wherein the non-endogenous receptor is a chimeric receptor.

38. The method of claim 37, wherein the chimeric receptor is a chimeric antigen receptor.

39. The method of claim 36, wherein the non-endogenous receptor is a non-natural T-cell receptor.

40. The method of claim 35, wherein the non-endogenous gene product comprises one or more non-endogenous receptors, one or more cytokines, one or more chemokines, one or more enzymes, or a combination thereof.

41. The method of any one of the preceding claims, wherein the immune cells are modified to have disruption of expression of one or more endogenous genes in the cells.

42. The method of any one of claims 12-41, wherein the cord blood cell viability is greater than 98% or 99%, the TNC recovery is greater than 76.3%, and the NRBC content is greater than 7.5×10⁷ or 8.0×10⁷.

43. The method of any one of the preceding claims, wherein the cord blood is derived from a fetus or infant at less than or equal to 39 weeks of gestational age, the viability of cord blood cells following thawing is greater than or equal to 86.5%, the expansion of the NK cells between days 0 and 6 in culture is greater than or equal to 7-fold, and the expansion of the NK cells between days 6 and 15 in culture is greater than or equal to 10⁵-fold.

44. A cord blood composition identified by any one of the methods of claims 1-43.

45. The composition of claim 44, comprised in a pharmaceutically acceptable carrier.

46. The composition of claim 44 formulated with one or more cryoprotectants.

47. A composition comprising a population of immune cells derived from the method of any one of claims 1-43.

48. A method of predicting efficacy of immune cells for therapy, comprising
measuring one or more cord blood compositions having not been frozen for the following or considering one or more of the following:
(a) cord blood cell viability;
(b) optionally total mononuclear cell (TNC) recovery; and
(c) nucleated red blood cell (NRBC) content;
(d) weight of the baby from which the cord blood is derived;
(e) race of the biological mother and/or biological father of the baby from which the cord blood is derived;
(f) optionally gestational age of the baby from which the cord blood is derived;
(g) optionally intra utero collection of the cord blood;
(h) optionally a biologically male baby from which the cord blood is derived;
(i) optionally a volume of the cord blood collected;
(j) optionally the number of cells of the extracted cord blood that are CD34+;
wherein the immune cells are efficacious for therapy when the cord blood composition comprises one or more of the following characteristics:
(a) cord blood cell viability greater than or equal to 98% or 99%;
(b) total mononuclear cell (TNC) recovery is greater than or equal to 76.3%; and
(c) nucleated red blood cell (NRBC) content less than or equal to 7.5×10⁷ or 8.0×10⁷
(d) weight of the baby from which the cord blood is derived is greater than 3650 grams;
(e) race of the biological mother and/or biological father of the baby from which the cord blood is derived is Caucasian;
(f) optionally gestational age of the baby from which the cord blood is derived is less than or equal to 38 weeks;
(g) optionally intra utero collection of the cord blood;
(h) optionally a biologically male baby from which the cord blood is derived;
(i) optionally a volume of the cord blood collected and the anticoagulant is <about 120, 115, 110, 100, 90, 80, 70, 60, or 50 mL;
(j) optionally the number of cells of the extracted cord blood are CD34+ is >0.4%.

49. The method of claim 48, further comprising the step of freezing the one or more blood compositions.

50. The method of claim 49, further comprising measuring upon thawing (d) cytotoxicity of immune cells derived from the cord blood composition.

51. The method of claim 50, wherein the cytotoxicity is greater than or equal to 66.7%.


SEQUENCE LISTING (3 sequences)

SEQ ID NO: 1 - DNA, 20 bp, Artificial Sequence
  Synthetic polynucleotide
gacaggcacaatagaaacaa

SEQ ID NO: 2 - DNA, 20 bp, Artificial Sequence
  Synthetic polynucleotide
tgagttcccaacttcattag

SEQ ID NO: 3 - DNA, 20 bp, Artificial Sequence
  Synthetic polynucleotide
gcgggacatgttcaccctgg